(19) United States
(12) Patent Application Publication
(10) Pub. No.: US 2006/0099610 A1
(43) Pub. Date: May 11, 2006

(54) Method and kit for detecting a risk of acute myocardial infarction

(75) Inventors: Jukka T. Salonen, Kuopio (FI); Juha-Matti Aalto, Siilinjarvi (FI); Ricardo Fuentes, Siilinjarvi (FI); Outi Kontkanen, Kuopio (FI); Mia Pirskanen, Kuopio (FI); Pekka Uimari, Kuopio (FI)

Correspondence Address: BIRCH STEWART KOLASCH & BIRCH, PO BOX 747, FALLS CHURCH, VA, 22040-0747, US

(73) Assignee: Oy Jurilab Ltd.

(21) Appl. No.: 11/143,642

(22) Filed: Oct. 11, 2005

(30) Foreign Application Priority Data
Oct. 15, 2004 (FI) 20041340

(51) Int. Cl. C12Q 1/68

(52) U.S. Cl. 435/6

(57) ABSTRACT

Genes, SNP markers and haplotypes of susceptibility or predisposition to CHD such as AMI are disclosed. Methods for diagnosis, prediction of clinical course and efficacy of treatments for AMI using polymorphisms in the AMI risk genes are also disclosed. The genes, gene products and agents of the invention are also useful for monitoring the effectiveness of prevention and treatment of AMI and CHD. Kits are also provided for the diagnosis, selecting treatment and assessing prognosis of CHD and AMI.

### BACKGROUND OF THE INVENTION

1. Field of the Invention

The present invention relates generally to the field of diagnosis of coronary heart disease (CHD) such as acute myocardial infarction (AMI). More particularly, it provides a method of diagnosing or detecting a predisposition or propensity or susceptibility for AMI. Specifically, the invention is directed to a method that comprises the steps of providing a biological sample of the subject to be tested and detecting the presence or absence of one or several genomic single nucleotide polymorphism (SNP) markers in the biological sample. Furthermore, the invention utilises both genetic and phenotypic information as well as information obtained by questionnaires to construct a score that provides the probability of developing AMI. In addition, the invention provides a kit to perform the method. The kit can be used to set an etiology-based diagnosis of AMI for targeting of treatment and preventive interventions, such as dietary advice as well as stratification of the subject in clinical trials testing drugs and other interventions. The invention also relates to a method for the treatment of CHD and AMI.

2. Description of Related Art

Public Health Significance of CVD and CHD

Cardiovascular Diseases (CVD) (ICD/10 codes I00-I99, Q20-Q28) include ischemic (coronary) heart disease (CHD), hypertensive diseases, cerebrovascular disease (stroke) and rheumatic fever/rheumatic heart disease, among others (AHA, 2004). In terms of morbidity, mortality and cost CHD is the most important disease group of CVD. CHD (ICD/10 codes I20-I25) includes acute myocardial infarction (AMI), other acute ischemic (coronary) heart disease, angina pectoris; atherosclerotic cardiovascular disease and all other forms of chronic ischemic heart disease (AHA, 2004). Here, acute coronary events, though not technically AMI, are included under the term “AMI”. AMI and angina pectoris are often caused by coronary atherosclerosis, but not always. Other, often contributory pathophysiologies include coronary thrombosis and contriction or contraction and severe arrhythmias. These may cause an AMI also without coronary narrowing by atherosclerosis.

In 2001 an estimated 16.6 million—or one-third of total global deaths—resulted from the various forms of CVD (7.2 million due to CHD, 5.5 million to cerebrovascular disease, and an additional 3.9 million to hypertensive and other heart conditions). At least 20 million people survive heart attacks and strokes every year, a significant proportion of them requiring costly clinical care, which puts a huge burden on long-term care resources. It is necessary to recognize that CVD are devastating to both men, women and children (ADA, 2004).

Around 80% of all CVD deaths worldwide took place in developing, low and middle-income countries. It is estimated that by 2010, CVD will be the leading cause of death in both developed and developing countries. The rise in CVDs reflects a significant change in dietary habits, physical activity levels, and tobacco consumption worldwide as a result of industrialization, urbanization, economic development and food market globalization (WHO, 2004). This emphasizes the role of relatively modern environmental or behavioral risk factors. However, ethnic differences in the incidence and prevalence of CVD and the enrichment of CVD in families suggest that heritable risk factors play a major role.

In terms of disability measured in disability-adjusted life years (DALYs) CVD caused 9.7% of global DALYs, 20.4% of DALYs in developed countries and 8.3% of DALYs in the developing countries (Murray CJL and Lopez AD, 1997).

On the basis of data from the NHANES III study (1988-1994), it is estimated that in 2001, 64.4 million Americans were affected by some form of CVD, which corresponds to a prevalence of 22.6% (21.5% for males, 22.4% for females). Of these, 13.2 million had CHD (6.4% prevalence).

The cost of CVD in the United States in 2004 is estimated at $368.4 billion ($133.2 billion for CHD, $53.6 billion for stroke, $55.5 billion for hypertensive disease). This figure includes health expenditures (direct costs) and lost productivity resulting from morbidity and mortality (indirect costs) (AHA, 2004).

It is important for the health care system to develop strategies to prevent AMI. Once AMI has manifested clinically, irreversible cell death and tissue damage starts to occur in the myocardial muscle. Unfortunately, the myocardial cells that die cannot be revived or replaced from a stem cell population. Also, a major part of the first clinical manifestations of CHD are sudden deaths. Therefore, it is better to prevent AMI from happening in the first place, i.e. primary prevention. Although we already know of certain clinical risk factors that increase AMI risk, there is an unmet medical need to define the genetic factors involved in AMI to more precisely define disease risk or susceptibility.

CHD: a Polygenic Disease

The etiology and pathophysiology of CVD are complexes, but it is known that major risk factors include unhealthy lifestyles and behaviours and a complex interaction between environmental and genetic factors. The four major CVD risk factors are habitual adverse dietary patterns (primarily high intake of cholesterol and saturated fats), habitual cigarette smoking, dyslipidaemia, indicated by adverse total cholesterol levels, and blood pressure above optimal level (Stamler J et al, 1998). Other well established CVD risk factors are age, male gender, obesity, physical inactivity and diabetes. The role of other emerging risk factors for CVD—thrombogenic factors, homocysteine, markers of inflammation, infection and genetic factors—in risk prediction and management is not established (Wood D, 2001).

A positive family history of premature CHD predicts development of CHD independently of other major CVD risk factors (Sholtz R I et al, 1975; Heller R F and Kelson M C, 1983; Barrett-Connor E and Khaw K, 1984; Colditz G A et al, 1986; Hopkins P N et al, 1988; Myers R H et al, 1990; Colditz G A et al, 1009; Jousilahti P et al, 1996; Boer J M et al, 1999; Li R et al, 2000; Hawe E et al, 2003) and persons with a history of family premature CHD who are otherwise predicted to be at low risk by standard risk factors may have a substantial genetic component for disease development (Heller R F and Kelson M C, 1983; Myers R H et al, 1990). The risk ratio of AMI associated with positive premature CHD of either parent is 1.61 in men and 1.85 in women (Jousilahti P et al, 1996). Further support for the genetic contribution to disease risk comes from twin studies. Marenberg et al, 1994 showed a high concordance for age of onset of CHD. Among men, the relative hazard of death from CHD when one's twin died of CHD before the age of 55 years, as compared with the hazard when one's twin did not die before 55, was 8.1 for monozygotic twins and 3.8 for dizygotic twins. Among women, when one's twin died of CHD before the age of 65 years, the relative hazard was 15.0 for monozygotic twins and 2.6 for dizygotic twins.

At the molecular level, atherosclerosis is a time dependent, multistep process involving the interaction of many different key pathways, including lipoprotein metabolism (Chisolm G M and Steinberg D, 2000), lipoprotein oxidation (Salonen J T et al, 1992), coagulation (Tremoli E et al, 1999) and inflammation (Ross R. 1999). Gene mutations in any of these pathways will only provide a partial contribution to risk. Intermediate phenotypes such as hypertension, diabetes, smoking and obesity interact to modulate risk as will do gene-gene and gene-environment interactions (Stephens J W and Humphries S E, 2003). Not all CHD is polygenic in nature; an exception is familial hypercholesterolaemia (FH) (Civeira F, 2004). The responsible mutations causing FH can now be screened for in high-risk individuals to allow early identification and to target early therapy.

In addition to coronary atherosclerosis, CHD and AMI may be caused by other mechanisms such as thrombosis, vasoconstriction and arrhythmias. Like atherosclerosis, also thrombosis can have many pathways. The role of platelet function, the coagulation and fibrinolytic systems is expected to be larger in coronary thrombosis than atherosclerosis. In addition, etiologies of atherosclerosis and thrombosis interact with each other.

Unlike the rare and severe genetic defects that cause monogenic diseases, the genetic factors that modulate the individual susceptibility to multifactorial diseases such as CVD are common, functionally different, forms of gene polymorphisms, which generally have a modest effect at an individual level but, because of their high carrier frequency in the population, can be associated with a high population attributable risk. Environmental factors can reveal or facilitate the phenotypic expression of such AMI risk genes.

Although more than a hundred putative gene associations to CHD have been reported, only a handful have been widely replicated (Fuentes R M, 2004, unpublished review). The association of APOA4 (Rewers M et al, 1994; Wong W M et al, 2003), APOB (Chiodini B D et al, 2003, meta-analysis), APOE (Eichner J E et al, 1993; Nakai K et al, 1998; Inbal a et al, 1999; Brscic E et al, 2000; Humphries S E et al, 2001; Baroni M G et al, 2003; Kumar P et al, 2003), F2 (Kim R J and Becker R C, 2003; Burzotta F et al, 2004, both meta-analyses), F5 (Kim R J and Becker R C, 2003, meta-analysis), IL6 (Rundek T et al, 2002; Georges J L et al, 2001; Humphries S E et al, 2001), MMP3 (Terashima M et al, 1999; Rundek T et al, 2002; Beyzade S et al, 2003; NOS3 (Shimasaki Y et al, 1998; Hibi K et al, 1998; Hingorani A D et al, 1999; Park J E et al, 2000; Cine N et al, 2002), PON1 (Serrato M and Marian A J, 1995; Sanghera D K et al, 1997; Salonen J T et al, 1999; Senti M et al, 2001), SERPINE1 (Pastinen T et al, 1998; Gardemann A et al, 1999; Ardissino D et al, 1999; Mikkelsson J et al, 2000; Fu L et al, 2001; Zhan M et al, 2003), and THBD (Norlund L et al, 1997; Wu K K et al, 2001; Li Y H et al, 2002; Park H Y et al, 2002; Chao T H et al, 2004) have been reproduced by independent groups. Interactions between IL6 and MMP3 (Rauramaa R et al, 2000) and between smoking and APOE (Humphries S E et al, 2001), F5 (Holm J et al, 1999), IL6 (Humphries S E et al, 2001), MMP3 (Humphries S E et al, 2002) and PON1 (Sen-Banerjee S et al, 2000) as examples of gene-gene and gene-environment interactions affecting the risk of CHD have also been reported.

Pathophysiology of Coronary Atherosclerosis

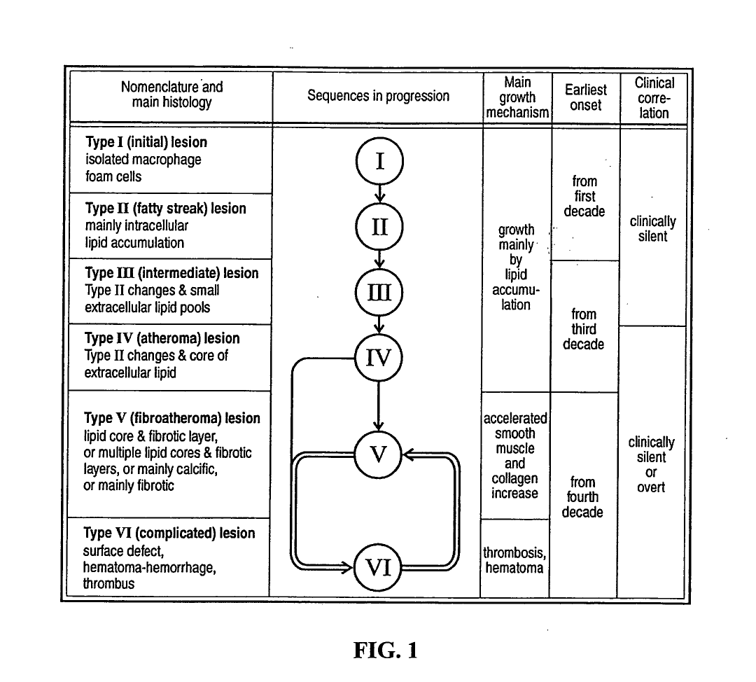

Progression of human atherosclerotic lesions: Human atherosclerotic lesions from the coronary arteries and the aorta can be obtained for study as specimens during therapeutic interventions or at autopsy of persons who died suddenly of causes other than disease. According to current histological criteria atherosclerosis in humans may be divided into two broad categories of lesions: minimal lesions and advanced lesions (Stary H C et al, 1994; Stary H C et al, 1995; Stary H C, 2000). In each of these broad categories three characteristic types of lesions are distinguished: types I (initial), II (fatty streak) and III (intermediate, preatheroma) in the minimal lesions; and types IV (atheroma), V (fibroatheroma) and VI (complicated) in the advanced lesions (FIG. 1). The contiguous nature of the histological changes and the time of life at which a specific change predominates indicate that each represents a gradation or stage in a temporal sequence.

Each type of minimal lesion is focal and relatively small, and contains abnormal accumulations of lipoproteins and cholesterol esters. Increased numbers of cells, mainly macrophages, and accumulations of lipid droplets, mainly within macrophages, can be demonstrated microscopically. Changes in the composition of the matrix and disruption of the intimal architecture are minimal or absent. The media adjacent to the lesions is not diseased, nor is the adventitia affected (Stary H C et al, 1994). Atherosclerotic lesions are considered advanced when accumulations of lipid, cells, and matrix components, including minerals, are associated with structural disorganization, repair and thickening of the intima, as well as deformity of the arterial wall (Stary H C et al, 1995).

It has been found that the arterial intima is thicker in highly susceptible locations from birth. This process is called adaptive intimal thickening, which develop in response to normal asymmetries in fluid mechanical forces to maintain an optimal flow equally at all points along the course of an artery. The thickenings begin to develop in foetal life and are found in everyone at birth (Stary H C et al, 1992). A thick intima is seen at and near bifurcations of arteries and at the mouths of small branch vessels, where it is focal and eccentric. The initial accumulations of lipid and macrophage foam cell in early life are more prominent in a subset of adaptive intimal thickenings. A distinct fluid mechanical force in such locations is low shear stress (Stary H C et al, 1992). In regions of low shear, circulating plasma particles are in longer contact with the endothelial surface. This enhances the frequency with which particles enter the intima. When plasma is too rich in lipoprotein, it accumulates most in these locations. If atheromas are present in later life, they are found here first.

Type I and II lesions are the only ones that occur in infants and children, although they also occur in adults. Type III lesions may evolve soon after puberty and type IV lesions are frequent from the third decade onward. After the third decade of life, lesions of type V and VI composition begin to appear. In middle-aged and older persons, these often become the predominant lesion types (Stary H C et al, 1995; Stary H C, 2000). Type V and VI lesions develop and progress by mechanisms that are, for the most part, different from and superimposed on the continuing lipid accumulation that produced lesion types I through IV (Stary H C et al, 1994; Stary H C et al, 1995; Steinberg D and Lewis A, 1997; Chisolm G M and Steinberg D, 2000). In type IV lesions disarrangement of intimal structure is caused almost solely by an extensive accumulation of extracellular lipid localized in the deep intima (the lipid core). In type V lesions the intima is thickened by substantial reparative fibrous tissue layers. Surface defects, hematoma and thrombotic deposits (type VI lesions) further damage, deform and thicken the intima and accelerate the conversion from clinically silent to overt disease.

The clinical significance of lesion types I, II, and III lies in their role as the silent precursors of possible future disease and their potential for reversibility. Recognition of the period of life in which type III lesions begin should lead to concentrated preventive measures at, or preferably before, that age (Stary H C et al, 1995; Stary H C, 2000). Morbidity and mortality from atherosclerosis is largely due to type IV and type V lesions in which disruptions of the lesion surface, hematoma or hemorrhage, and thrombotic deposits have developed (type VI lesions), which have their clinical correlate as acute ischemic coronary syndromes (Davies M J, 1990; Libby P, 1995).

Molecular biology of human atherosclerotic lesions: The earliest changes that precede the formation of lesions of atherosclerosis take place in the endothelium. These changes include increased endothelial permeability to lipoproteins and other plasma constituents, which is mediated by nitric oxide, prostacyclin, platelet-derived growth factor, angiotensin II, and endothelin; up-regulation of leukocyte adhesion molecules, including L-selectin, integrins, and platelet-endothelial-cell adhesion molecule 1, and the up-regulation of endothelial adhesion molecules, which include E-selectin, P-selectin, intercellular adhesion molecule 1, and vascular-cell adhesion molecule 1; and migration of leukocytes into the artery wall, which is mediated by oxidized low-density lipoprotein, monocyte chemotactic protein 1, interleukin-8, platelet-derived growth factor, macrophage colony-stimulating factor, and osteopontin (Ross R, 1999).

Fatty streaks initially consist of lipid-laden monocytes and macrophages (foam cells) together with T lymphocytes. Later they are joined by various numbers of smooth-muscle cells. The steps involved in this process include smooth-muscle migration, which is stimulated by platelet-derived growth factor, fibroblast growth factor 2, and transforming growth factor β; T-cell activation, which is mediated by tumor necrosis factor α, interleukin-2, and granulocyte-macrophage colony-stimulating factor; foam-cell formation, which is mediated by oxidized low-density lipoprotein, macrophage colony-stimulating factor, tumor necrosis factor α, and interleukin-1; and platelet adherence and aggregation, which are stimulated by integrins, P-selectin, fibrin, thromboxane A2, tissue factor, and the factors described above responsible for the adherence and migration of leukocytes (Ross R, 1999).

As fatty streaks progress to intermediate and advanced lesions, they tend to form a fibrous cap that walls off the lesion from the lumen. This represents a type of healing or fibrous response to the injury. The fibrous cap covers a mixture of leukocytes, lipid, and debris, which may form a necrotic core. These lesions expand at their shoulders by means of continued leukocyte adhesion and entry caused by the same factors as those participating in endothelial dysfunction and fatty-streak formation. The principal factors associated with macrophage accumulation include macrophage colony-stimulating factor, monocyte chemotactic protein 1, and oxidized low-density lipoprotein. The necrotic core represents the results of apoptosis and necrosis, increased proteolytic activity, and lipid accumulation. The fibrous cap forms as a result of increased activity of platelet-derived growth factor, transforming growth factor β, interleukin-1, tumor necrosis factor α, and osteopontin and of decreased connective-tissue degradation (Ross R, 1999).

Rupture of the fibrous cap or ulceration of the fibrous plaque can rapidly lead to thrombosis and usually occurs at sites of thinning of the fibrous cap that covers the advanced lesion. Thinning of the fibrous cap is apparently due to the continuing influx and activation of macrophages, which release metalloproteinases and other proteolytic enzymes at these sites. These enzymes cause degradation of the matrix, which can lead to hemorrhage from the vasa vasorum or from the lumen of the artery and can result in thrombus formation and occlusion of the artery (Ross R, 1999).

Genome-Wide Scan Studies in CHD

The use of genetic marker maps is based on the concept that a gene marker will segregate from a generation to another with a locus causing CHD/AMI. The sections of chromosome inherited in a family are large, typically cMs. Theoretically, a microsatellite marker map should contain 5,000-10,000 markers to cover the entire genome. In practice, genome-wide scans (GWS) family studies have used typically 400 markers or so, which is insufficient to find the majority of disease genes.

A total of 6 GWS studies in CHD (Pajukanta P et al, 2000; Francke S et al, 2001; Broeckel U et al, 2002; Harrap S B et al, 2002; Wang Q et al, 2004; Fox C S et al, 2004) and one meta-analysis including data from 4 original GWS (Chiodini B D and Lewis C M, 2003) have been

TABLE 1
Genome wide scans carried out previously in coronary heart disease.

| | Fox C S | Wang Q | Chiodini B D | Harrap S B | Broeckel U | Francke S | Pajukanta P |
|---|---|---|---|---|---|---|---|
| | et al. | et al. | & Lewis C M | et al. | et al. | et al. | et al. |
| Publication | February, | February, | October, | May, | February, | November, | December, |
| year | 2004 | 2004 | 2003 | 2002 | 2002 | 2001 | 2000 |
| Population | US | US | DE, FI, | AU | DE | MU | FI |
| | | | AU, MU | | | | |
| Individuals | 1225 | 1613 | ND | 213 | 1406 | 535 | 364 |
| Families | 311 | 428 | 807 | 61 sib | 513 | 99 | 156 |
| | | | | pairs | | | |
| Phenotype | ICA/CCA | CAD/AMI | CHD | ACS | AMI | CHD | CHD |
| | IMT | | | | | | |
| Marker | ˜400 | 408 | 303-400 | 400 | 394 | 403 | 303-385 |
| density | | | | | | | |
| Chromosome | | | | | | | |
| 1 | | 1p36, 1p21 | | | | | |
| 2 | | 2p11 | 2q34-37 | 2q36-q37 | | | 2q22-q23 |
| 3 | | 3p24 | 3q26-q27 | 3q26-q27 | | 3q27-q29 | |
| 4 | | 4p16, 4q31, | | | | | |
| | | 4q32 | | | | | |
| 5 | | 5p15, 5p14, | | | | | |
| | | 5p12, | | | | | |
| | | 5q14, 5q22 | | | | | |
| 7 | | 7q22 | | | | | |
| 8 | | | | | | 8q23 | |
| 9 | | 9p21 | | | | | |
| 10 | | 10p14, 10q21 | | | | 10q23 | |
| 11 | | 11q12, 11q13, | | | | | |
| | | 11q23 | | | | | |
| 12 | 12q24 | 12q24 | | | | | |
| 13 | | 13q32 | | | | | |
| 14 | | 14q22, 14q24 | | | 14q32 | | |
| 16 | | 16q11 | | | | 16p13 | |
| 20 | | 20p12 | | 20q11-q13 | | | |
| 21 | | 21q22 | | | | | |
| X | | | | | | | Xq23-q25 |

Citogenic band locations have been updated according to genome build 34; CAD, coronary artery disease; CHD, coronary heart disease; AMI, acute myocardial infarction; ACS, acute coronary syndrome; ICA IMT, internal carotid artery intimal medial thickness; CCA IMT, common carotid artery intimal medial thickness; US, United States; FI, Finland; DE, Germany; AU, Australia; MU, Mauritius

reported to date. Most of these studies have considered chronic CHD survivors, thus are prone to survival bias (see table 1).

Pajukanta P et al, 2000, found in a linkage study with just over 300 microsatellite markers in 156 families (364 subjects) two large linkage regions in 2q22-q23 and Xq23-q25. The initial linkage regions were 40 cM and 30 cM, respectively. Some fine mapping was done with a very limited number of markers with average 2.5 cM marker interval. Candidate genes in the strongest linkage regions were speculated.

In a larger linkage study using 408 microsatellite markers in 1613 subjects from 428 American mainly Caucasian extended families, Wang Q et al, 2004, found a novel coronary artery disease (CAD)/AMI susceptibility locus in a very large, 32 cM, region in 1p36.

Other GWS studies have found linkage in 2q36-q37, 3q26-q27 and 20q11-q13 for acute coronary syndrome (ACS)( Harrap S B et al, 2002), 12q24 for carotid intimal medial thickness (Fox C S et al, 2004), 14q32 for AMI (Broeckel U et al, 2002) and 3q27-q29, 8q23, 10q23 and 16p13 for CHD (Francke S et al, 2001). A meta-analysis by Chiodini B D and Lewis C M, 2003, concluded that the genetic regions 2q34-q37 and 3q26-q27 might contain AMI risk genes for CHD.

In summary, previous findings of GWS linkage studies in CHD, AMI or atherosclerosis are inconsistent. Each and every study has detected linkage signals in different chromosomal regions. As the number of gene markers has been small, the regions identified have been large, of many cMs.

Opportunity for Population Genetics

Previous medical research concerning the genetic etiology of CHD and AMI has been based to a large extent on retrospective case-control and family studies in humans and studies in genetically modified animals. As recognized only recently, retrospective case-control studies are prone to survival and selection biases, and they have produced a myriad of biased findings concerning a large number of candidate genes. A commonly used approach is to compare gene expression between affected and unaffected persons. Gene expression studies, which are mostly cross-sectional, cannot however separate cause and consequence. Findings from animal models concerning CHD cannot be generalised to humans, as the pathophysiology in humans is unique. The uselessness of the animal studies is the main reason why genetic epidemiologic studies are the most important means in the clarification of genetic etiologies of human diseases.

Prospective cohort studies in humans overcome these problems. Developments in GWS and sequencing technology and methods of data analysis render now possible the attempt to identify liability genes in complex, multifactorial traits, and to dissect out with new precision the role of genetic predisposition and environment/life style factors in these disorders. Genetic and environmental effects vary over the life span, and only longitudinal studies in genetically informative data sets permit the study of such effects. A major advantage of population genetics approaches in disease gene discovery over other methodologies is that it will yield diagnostic markers which are valid in humans.

Identification of genes causing the major public health problems such as CHD is now enabled by the following recent advances in molecular biology, population genetics and bioinformatics: 1. the availability of new genotyping platforms that will dramatically lower operating cost and increase throughputs; 2. the application of genome scans using dense marker maps (>100.000 markers); 3. data analysis using new powerful statistical methods testing for linkage disequilibrium using haplotype sharing analysis, and 4. the recognition that a smaller number of genetic markers than previously thought is sufficient for genome scans in genetically homogeneous populations.

Traditional GWS using microsatellite markers with linkage analyses have not been successful in finding genes causing common diseases. The failure has in part been due to too small a number of genetic markers used in GWS, and in part due to too heterogeneous study populations. With the advancements of the human genome project and genotyping technology, the first dense marker maps have recently become available for mapping the entire human genome. The microarrays used by Jurilab include probes for over 100,000 single nucleotide polymorphism (SNP) markers. These SNPs form a marker map covering, for the first time, the entire genome tightly enough for the discovery of most disease genes causing AMI.

Genetic Homogeneity of the East Finland Founder Population

Finns descend from two human immigration waves occurring about 4,000 and 2,000 years ago. Both Y-chromosomal haplotypes and mitochondrial sequences show low genetic diversity among Finns compared with other European populations and confirm the long-standing isolation of Finland (Sajantila A et al, 1996). During King Gustavus of Vasa (1523-1560) over 300 years ago, internal migrations created regional subisolates, the late settlements (Peltonen L et al, 1999). The most isolated of these are the East Finns.

The East Finnish population is the most genetically-homogenous population isolate known that is large enough for effective gene discovery program. The reasons for homogeneity are: the young age of the population (fewer generations); the small number of founders; long-term geographical isolation; and population bottlenecks because of wars, famine and fatal disease epidemics.

Owing to the genetic homogeneity of the East Finland population there are fewer mutations and haplotypes in important disease predisposing genes and the affected individuals share similar genetic background. Because of the stronger linkage disequilibrium (LD) fewer SNPs and fewer subjects are needed for GWS studies.

### BRIEF DESCRIPTION OF THE DRAWING

The sole drawing illustrates the pathways in the evolution and progression of human atherosclerotic lesions (from Stary H C et al, 1995, without modification).

### SUMMARY OF THE INVENTION

The present invention relates to single nucleotide polymorphism (SNP) markers, combinations of such markers and haplotypes associated with altered risk of AMI and other coronary events and genes associated with AMI or other coronary events within or close to which the said markers or haplotypes formed by some of these markers are located. The said SNP markers may be associated either with increased AMI risk or reduced AMI risk i.e. protective of AMI. The “prediction” or risk implies here that the risk is either increased or reduced.

Thus the present invention provides individual SNP markers associated with AMI and combinations of SNP markers and haplotypes in genetic regions associated with AMI, genes previously known in the art, but not known to be associated with AMI, methods of estimating susceptibility or predisposition of an individual to AMI, as well as methods for prediction of clinical course and efficacy of treatments for AMI using polymorphisms in the AMI risk genes. Accordingly the present invention provides novel methods and compositions based on the disclosed AMI associated SNP markers, combinations of SNP markers, haplotypes and genes.

The invention further relates to a method for estimating susceptibility or predisposition of an individual to AMI comprising the detection of the presence of SNP markers and haplotypes or an alteration in expression of an AMI risk gene set forth in tables 3 through 11, as well as alterations in the polypeptides encoded by the said AMI risk genes. The alterations may be quantitative, qualitative, or both.

The invention yet further relates to a method for estimating susceptibility or predisposition of an individual to AMI. The method for estimating susceptibility or predisposition of an individual to AMI is comprised of detecting the presence of at-risk haplotypes in an individual's nucleic acid.

The invention further relates to a kit for estimating susceptibility to AMI in an individual comprising wholly or in part: amplification reagents for amplifying nucleic acid fragments containing SNP markers, detection reagents for genotyping SNP markers and interpretation software for data analysis and risk assessment.

In one aspect, the invention relates to methods of diagnosing a predisposition to AMI. The methods of diagnosing a predisposition to AMI in an individual include detecting the presence of SNP markers predicting AMI, as well as detecting alterations in expression of genes which are associated with said markers. The alterations in expression can be quantitative, qualitative, or both.

A further object of the present invention is a method of identifying the risk of AMI and CHD by detecting SNP markers in a biological sample of the subject. The information obtained from this method can be combined with other information concerning an individual, e.g. results from blood measurements, clinical examination and questionnaires. The blood measurements include but are not restricted to the determination of plasma or serum cholesterol and high-density lipoprotein cholesterol. The information to be collected by questionnaire includes information concerning gender, age, family and medical history such as the family history of CHD and diabetes. Clinical information collected by examination includes e.g. information concerning height, weight, hip and waist circumference, systolic and diastolic blood pressure, and heart rate.

The methods of the invention allow the accurate diagnosis of AMI at or before disease onset, thus reducing or minimizing the debilitating effects of AMI. The method can be applied in persons who are free of clinical symptoms and signs of CHD, in those who already have clinical CHD, in those who have family history of CHD or in those who have elevated level or levels of risk factors of AMI or CHD.

The invention further provides a method of diagnosing susceptibility to AMI in an individual. This method comprises screening for at-risk haplotypes that predict AMI that are more frequently present in an individual susceptible to AMI, compared to the frequency of its presence in the general population, wherein the presence of an at-risk haplotype is indicative of a susceptibility to AMI. The “at-risk haplotype” may also be associated with a reduced rather than increased risk of AMI. An “at-risk haplotype” is intended to embrace one or a combination of haplotypes described herein over the markers that show high correlation to AMI. Kits for diagnosing susceptibility to AMI in an individual are also disclosed.

Those skilled in the art will readily recognize that the analysis of the nucleotides present in one or several of the SNP markers of this invention in an individual's nucleic acid can be done by any method or technique capable of determining nucleotides present in a polymorphic site. As it is obvious in the art the nucleotides present in SNP markers can be determined from either nucleic acid strand or from both strands.

The major application of the current invention involves prediction of those at higher risk of developing AMI and other acute coronary events. Diagnostic tests that define genetic factors contributing to AMI might be used together with or independent of the known clinical risk factors to define an individual's risk relative to the general population. Better means for identifying those individuals at risk for AMI should lead to better preventive and treatment regimens, including more aggressive management of the current clinical risk factors such as cigarette smoking, hypercholesterolemia, elevated LDL cholesterol, low HDL cholesterol, hypertension and elevated blood pressure, diabetes mellitus, glucose intolerance, insulin resistance and the metabolic syndrome, obesity, lack of physical activity, and inflammatory components as reflected by increased C-reactive protein levels or other inflammatory markers. Information on genetic risk may be used by physicians to help convince particular patients to adjust life style (e.g. to stop smoking, reduce caloric intake, to increase exercise).

A further object of the invention is to provide a method for the selection of human subjects for studies testing anticoronary and antihypertensive effects of drugs.

Another object of the invention is a method for the selection of subjects for clinical trials testing anticoronary and antihypertensive drugs.

Still another object of the invention is to provide a method for prediction of clinical course and efficacy of treatments for AMI using polymorphisms in the AMI risk genes. The genes, gene products and agents of the invention are also useful for treating other related clinical or coronary events such as angina pectoris, for monitoring the effectiveness of their treatment, and for drug development. Kits are also provided for the diagnosis, treatment and prognosis of CHD and AMI.

A further object of the invention is a method for treating CHD in a subject with CHD or treating AMI in a subject with AMI by influencing the DNA sequence, expression or proteins of any of the genes of the invention in a human or animal subject. A related object of the invention is a method for preventing the onset of CHD in a subject or preventing AMI in a subject by influencing the DNA sequence, expression or proteins of any of the genes of the invention in a human or animal subject.

### DETAILED DESCRIPTION OF THE INVENTION

Representative Target Population

An individual at risk of AMI is an individual who has at least one risk factor, such as family history of AMI, cigarette smoking, hypercholesterolemia, elevated LDL cholesterol, low HDL cholesterol, hypertension and elevated blood pressure, diabetes mellitus, glucose intolerance, insulin resistance and the metabolic syndrome, obesity, lack of physical activity, elevated inflammatory marker, and an at-risk allele or haplotype with one or several AMI risk SNP markers.

In another embodiment of the invention, an individual who is at risk of AMI is an individual who has a risk-increasing allele in an AMI risk gene, in which the presence of the polymorphism is indicative of a susceptibility to AMI. The term “gene,” as used herein, refers to an entirety containing all regulatory elements located both upstream and downstream as well as within of a polypeptide encoding sequence, 5′ and 3′ untranslated regions of mRNA and the entire polypeptide encoding sequence including all exon and intron sequences (also alternatively spliced exons and introns) of a gene.

Assessment for At-Risk Alleles and At-Risk Haplotypes

The genetic markers are particular “alleles” at “polymorphic sites” associated with AMI. A nucleotide position at which more than one sequence is possible in a population, is referred to herein as a “polymorphic site”. Where a polymorphic site is a single nucleotide in length, the site is referred to as a SNP. For example, if at a particular chromosomal location, one member of a population has an adenine and another member of the population has a thymine at the same position, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a thymine allele.

Typically, a reference nucleotide sequence is referred to for a particular gene. Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences.

Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, aninoacid change or abnormal mRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by an AMI susceptibility gene. For example, a nucleotide change resulting in a change in polypeptide sequence can alter the physiological properties of a polypeptide dramatically by resulting in altered activity, distribution and stability or otherwise affect on properties of a polypeptide.

Alternatively, nucleotide sequence variants can result in changes affecting transcription of a gene or translation of its mRNA. A polymorphic site located in a regulatory region of a gene may result in altered transcription of a gene e.g. due to altered tissue specifity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of an AMI susceptibility gene.

A “haplotype,” as described herein, refers to any combination of genetic markers (“alleles”), such as those set forth in tables 4, 5, 6, 7, 8, 10 and 11. A haplotype can comprise two or more alleles.

As it is recognized by those skilled in the art the same haplotype can be described differently by determining the haplotype defining alleles from different strands e.g. the haplotype rs834485, rs856283, rs1260817, rs1260772 (T G C A) described in this invention is the same as haplotype rs834485, rs856283, rs1260817, rs1260772 (A C G T) in which the alleles are determined from the other strand or haplotype rs834485, rs856283, rs1260817, rs1260772 (A G C A), in which the first allele is determined from the other strand.

The haplotypes described herein, e.g., having markers such as those shown in tables 4, 5, 6, 7, 8, 10 and 11 are found more frequently in individuals with AMI than in individuals without AMI. Therefore, these haplotypes have predictive value for detecting AMI or a susceptibility to AMI in an individual. Therefore, detecting haplotypes can be accomplished by methods known in the art for detecting sequences at polymorphic sites

It is understood that the AMI associated at-risk alleles and at-risk haplotypes described in this invention may be associated with other “polymorphic sites” located in AMI associated genes of this invention. These other AMI associated polymorphic sites may be either equally useful as genetic markers or even more useful as causative variations explaining the observed association of at-risk alleles and at-risk haplotypes of this invention to AMI.

In certain methods described herein, an individual who is at risk for AMI is an individual in whom an at-risk allele or an at-risk haplotype is identified. In one embodiment, the at-risk allele or the at-risk haplotype is one that confers a significant risk of AMI. In one embodiment, significance associated with an allele or a haplotype is measured by an odds ratio. In a further embodiment, the significance is measured by a percentage. In one embodiment, a significant risk is measured as odds ratio of at least about 1.2, including by not limited to: 0.1, 0.2, 0.3, 0.4, 0.5, 0.6, 0.7, 0.8, 0.9, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the allele or the haplotype, and often, environmental factors.

An at-risk haplotype in, or comprising portions of, the AMI risk gene, is one where the haplotype is more frequently present in an individual at risk for AMI (affected), compared to the frequency of its presence in a healthy individual (control), and wherein the presence of the haplotype is indicative of AMI or susceptibility to AMI.

In a preferred embodiment, the method comprises assessing in an individual the presence or frequency of SNPs in, comprising portions of, an AMI risk gene, wherein an excess or higher frequency of the SNPs compared to a healthy control individual is indicative that the individual has AMI, or is susceptible to AMI. See, for example, tables 4, 5, 6, 7, 8, 10 and 11 for SNPs that can form haplotypes that can be used as screening tools. These SNP markers can be identified in at-risk haploptypes. For example, an at-risk haplotype can include microsatellite markers and/or SNPs such as those set forth in tables 4, 5, 6, 7, 8, 10 and 11. The presence of the haplotype is indicative of AMI, or a susceptibility to AMI, and therefore is indicative of an individual who falls within a target population for the treatment methods described herein.

Consequently, the method of the invention is particularly directed to the detection of one or several of the SNP markers defining the following at-risk haplotypes indicative of AMI:
1) rs834485 (C/T), rs856283 (A/G), rs1260817 (A/C), rs1260772 (A/G) defining the haplotype T G C A;
2) rs1932818 (C/T), rs6663269 (C/G) defining the haplotype C C;
3) rs7605386 (C/G), rs9288697 (A/C) defining the haplotype G A;
4) rs3903306 (A/T), rs6432539 (C/T), rs1364703 (C/G), rs1966530 (A/G), rs5002908 (A/G) defining the haplotype A T G G A;
5) rs997274 (C/T), rs6788511 (A/C) defining the haplotype T C;
6) rs864391 (C/T), rs854202 (A/G), rs2014378 (A/G), rs952621 (A/G), rs1877960 (C/T) defining the haplotype T A A G C;
7) rs1445362 (A/G), rs10513252 (A/G), rs10513253 (C/T), rs4610179 (A/G) defining the haplotype A A T G;
8) rs682913 (C/T), rs725425 (A/C), rs1423260 (A/G) defining the haplotype T A G;
9) rs919740 (C/T), rs10515639 (A/G) defining the haplotype C A;
10) rs10499001 (C/T), rs4144270 (A/G), rs4515397 (A/G), rs10499000 (C/T) defining the haplotype T A G T;
11) rs1407658 (C/T), rs2025272 (G/T), rs9283864 (C/G) defining the haplotype C T G;
12) rs10503268 (A/C), rs1038062 (C/T), rs10503269 (A/G), rs1038058 (C/T) defining the haplotype A C G T;
13) rs10503616 (C/T), rs10503617 (C/T), rs2717719 (C/T), rs1488925 (A/G), rs2035681 (C/T) defining the haplotype C C C G C;
14) rs2994298 (C/T), rs7463074 (A/G), rs2975534 (C/T), rs2975533 (C/T) defining the haplotype C G T C;
15) rs10491744 (A/G), rs1543587 (A/G), rs1074789 (C/T), rs7025842 (C/T) defining the haplotype G G T T;
16) rs10491753 (A/C), rs13284133 (A/C), rs10491756 (G/T), rs10491757 (A/G) defining the haplotype A C T A;
17) rs1542750 (A/G), rs10501763 (C/T), rs10501764 (A/G), rs10501765 (C/T) defining the haplotype G T G C;
18) rs721346 (A/C), rs1400549 (C/T), rs503208 (C/G), rs7127296 (A/C) defining the haplotype A T C C;
19) rs10483879 (C/T), rs2885625 (A/G), rs6574333 (A/G) defining the haplotype C G A;
20) rs1049884 (A/T), rs1364256 (C/T), rs7185078 (C/T), rs7193075 (A/G) defining the haplotype A T T A;
21) rs10521277 (A/G), rs1019156 (A/G) defining the haplotype G G;
22) rs223128 (G/T), rs223154 (G/T), rs315499 (A/G), rs10512437 (A/G) defining the haplotype G T G G;
23) rs10153997 (C/T), rs10513998 (A/G), rs10514026 (T/G) defining the haplotype T A T;
24) rs799275 (C/T), rs5957801 (A/G), rs5909851 (A/G), rs10521707 (A/G) defining the haplotype T G G A;
25) rs764198 (A/G), rs10521816 (A/G), rs10521817 (C/T), rs3135496 (C/T) defining the haplotype G A C T;
26) rs623360 (C/G) and rs627069 (C/G) defining the haplotype C C;
27) rs7547716 (A/C), rs1891174 (C/T), and rs10494841 (A/G) defining the haplotype C T G;
28) rs10493353 (C/T), rs1323851 (G/T), rs834485 (C/T), rs856283 (A/G), and rs1260817 (A/C) defining the haplotype C G T G C;
29) rs3106653 (A/C), rs2961958 (A/T), rs2591169 (A/T), and rs10497144 (C/G) defining the haplotype A T A C;
30) rs10497192 (C/T), rs7605386 (C/G), and rs9288697 (A/C) defining the haplotype C G A;
31) rs1851328 (A/G), rs6711457 (C/T), rs10498053 (C/G), and rs6744504 (C/T) defining the haplotype G C G T;
32) rs9310496 (A/G), rs10510450 (A/G), rs4685356 (A/C), and rs10510452 (A/G) defining the haplotype A G C A;
33) rs10510660 (A/G), rs951973 (C/T), rs1487994 (G/T), rs10510661 (C/G), and rs6805290 (C/T) defining the haplotype A C T G C;
34) rs1018341 (G/T), rs953304 (C/G), rs10514726 (G/T) defining the haplotype G C G;
35) rs1355533 (C/T), rs1357287 (A/G), rs1403101 (C/T), and rs1356612 (C/T) defining the haplotype C G T C;
36) rs10513261 (C/T) and rs1881922 (C/T) defining the haplotype T C;
37) rs1023714 (A/T), rs2400502 (A/G), rs2400503 (C/T), rs10515605 (A/G), and rs7709159 (C/T) defining the haplotype A A C A C;
38) rs10515495 (A/G), rs4976445 (A/G), rs10491335 (A/G), and rs6878439 (A/G) defining the haplotype G A G G;
39) rs786135 (A/C), rs1357194 (C/T), rs9285412 (C/T), and rs9320498 (A/C) defining the haplotype A C C C (or nucleotides from the complementary strand);
40) rs10486559 (A/G), rs10486562 (A/G), rs757397 (C/T), rs735664 (C/T), and rs720659 (G/T) defining the haplotype A A T T T;
41) rs1961352 (A/C), rs2029832 (C/T), rs9741 (C/T), and rs10503616 (C/T) defining the haplotype C C T T;
42) rs10503555 (A/G), rs10503557 (C/T), rs2410361 (A/C), and rs10503561 (C/T) defining the haplotype G C C C;
43) rs260816 (C/G), rs260818 (A/C), rs361315 (A/G) defining the haplotype G C A (or nucleotides from the complementary strand);
44) rs1940357 (A/C), rs535908 (C/T), rs510628 (A/T) defining the haplotype C C A (or nucleotides from the complementary strand);
45) rs2706218 (C/T), rs897167 (C/T), rs10506333 (C/G), rs998401 (C/T), and rs3741673 (A/G) defining the haplotype T C G C G;
46) rs762721 (A/C), rs10491992 (C/G), rs10491993 (A/G), and rs2213177 (A/G) defining the haplotype C G A A;
47) rs760002 (A/C), rs10483259 (A/C), rs956163 (C/T), and rs10483261 (A/G) defining the haplotype C C T G;
48) rs2713961 (A/G), rs10512591 (A/G), rs2344989 (C/T), and rs2452932 (C/G) defining the haplotype A A T C;
49) rs10502579 (C/T), rs627346 (A/G), rs10502582 (A/T), and rs10502584 (A/G) defining the haplotype T A A G;
50) rs5949853 (A/T), rs4348668 (A/G), rs10521497 (C/T), rs707289 (A/G), and rs20369 (C/T) defining the haplotype A G A C;
51) rs764198 (A/G), rs10521816 (A/G), rs10521817 (C/T), and rs3135496 (C/T) defining the haplotype G A C T;
52) rs10521773 (C/T), rs2022565 (A/G), rs1569890 (A/G), rs5977614 (C/T), and rs10521774 (C/G) defining the haplotype C G G T G.
   Monitoring Progress of Treatment

The current invention also pertains to methods of monitoring the effectiveness of a treatment of AMI on the expression (e.g., relative or absolute expression) of one or more AMI risk genes. AMI susceptibility gene mRNA or polypeptide it is encoding or biological activity of the encoded polypeptide can be measured in a sample of peripheral blood or cells derived therefrom. An assessment of the levels of expression or biological activity of the polypeptide can be made before and during treatment with AMI therapeutic agents.

For example, in one embodiment of the invention, an individual who is a member of the target population can be assessed for response to treatment with an AMI inhibitor, by examining AMI risk gene encoding polypeptide biological activity or absolute and/or relative levels of AMI risk gene encoding polypeptide or mRNA in peripheral blood in general or specific cell subfractions or combination of cell subfractions.

In addition, variations such as haplotypes or mutations within or near (within one to hundreds of kb) of the AMI risk gene may be used to identify individuals who are at higher risk for AMI to increase the power and efficiency of clinical trials for pharmaceutical agents to prevent or treat AMI or its complications. The haplotypes and other variations may be used to exclude or fractionate patients in a clinical trial who are likely to have involvement of another pathway in their AMI in order to enrich patients who have pathways involved that are relevant regarding to the treatment tested and boost the power and sensitivity of the clinical trial. Such variations may be used as a pharmacogenetic test to guide selection of pharmaceutical agents for individuals.

Primers, Probes and Nucleic Acid Molecules

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Such probes and primers include polypeptide nucleic acids (Nielsen P E et al, 1991).

A probe or primer comprises a region of nucleic acid that hybridizes to at least about 15, for example about 20-25, and in certain embodiments about 40, 50 or 75, consecutive nucleotides of a nucleic acid of the invention, such as a nucleic acid comprising a contiguous nucleic acid sequence.

In preferred embodiments, a probe or primer comprises 100 or fewer nucleotides, in certain embodiments, from 6 to 50 nucleotides, for example, from 12 to 30 nucleotides. In other embodiments, the probe or primer is at least 70% identical to the contiguous nucleic acid sequence or to the complement of the contiguous nucleotide sequence, for example, at least 80% identical, in certain embodiments at least 90% identical, and in other embodiments at least 95% identical, or even capable of selectively hybridizing to the contiguous nucleic acid sequence or to the complement of the contiguous nucleotide sequence. Often, the probe or primer further comprises a label, e.g., radioisotope, fluorescent compound, enzyme, or enzyme co-factor.

Antisense nucleic acid molecules of the invention can be designed using the nucleotide sequences of tables 3-8, 10 and 11, and/or the complement of tables 3-8, 10 and 11, and/or a portion of tables 3-8, 10 and 11, and/or the complement of tables 3-8, 10 and 11, and/or a sequence encoding the amino acid sequences (wherein any one of these may optionally comprise at least one polymorphism as shown in tables 3-8, 10 and 11) and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid of interest).

The nucleic acid sequences of the AMI associated genes described in this invention can also be used to compare with endogenous DNA sequences in patients to identify genetic disorders (e.g., a predisposition for or susceptibility to AMI), and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Polyclonal and Monoclonal Antibodies

Polyclonal and/or monoclonal antibodies that specifically bind one form of the gene product but not to the other form of the gene product are also provided. Antibodies are also provided that bind a portion of either the variant or the reference gene product that contains the polymorphic site or sites. The term “antibody” as used herein refers to immunoglobulin molecules and immunologically active portions of immunoglobulin molecules, i.e., molecules that contain an antigen binding site that specifically binds an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′).sub.2 fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as known by those skilled in the art by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique (Kohler G and Milstein C, 1975), the human B cell hybridoma technique (Kozbor D et al, 1982), the EBV-hybridoma technique (Cole S P et al, 1994), or trioma techniques (Hering S et al, 1988). To produce a hybridoma an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (Bierer B et al, 2002). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide (Hayashi N et al, 1995; Hay B N et al, 1992; Huse W D et al, 1989; Griffiths A D et al, 1993). Kits for generating and screening phage display libraries are commercially available.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue such as blood as part of a test predicting the susceptibility to AMI or as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include .sup.125I, 131I, 35S or 3H.

Diagnostic Assays

The probes, primers and antibodies described herein can be used in methods of diagnosis of AMI or diagnosis of a susceptibility to AMI, as well as in kits useful for diagnosis of AMI or susceptibility to AMI or to a disease or condition associated with AMI.

In one embodiment of the invention, diagnosis of AMI or susceptibility to AMI (or diagnosis of or susceptibility to a disease or condition associated with AMI), is made by detecting one or several of at-risk alleles or at-risk haplotypes or a combination of at-risk alleles and at-risk haplotypes described in this invention in the subject's nucleic acid as described herein.

In one embodiment of the invention, diagnosis of AMI or susceptibility to AMI (or diagnosis of or susceptibility to a disease or condition associated with AMI), is made by detecting one or several of polymorphic sites which are associated with at-risk alleles or/and at-risk haplotypes described in this invention in the subject's nucleic acid. Diagnostically the most useful polymorphic sites are those altering the polypeptide structure of an AMI associated gene due to a frame shift; due to a premature stop codon, due to an aminoacid change or due to abnormal mRNA splicing. Nucleotide changes resulting in a change in polypeptide sequence in many cases alter the physiological properties of a polypeptide by resulting in altered activity, distribution and stability or otherwise affect on properties of a polypeptide. Other diagnostically useful polymorphic sites are those affecting transcription of an AMI associated gene or translation of it's mRNA due to altered tissue specifity, due to altered transcription rate, due to altered response to physiological status, due to altered translation efficiency of the mRNA and due to altered stability of the mRNA. The presence of nucleotide sequence variants altering the polypeptide structure of AMI associated genes or altering the expression of AMI associated genes is diagnostic for susceptibility to AMI.

For diagnostic applications, there may be polymorphisms informative for prediction of disease risk that are in linkage disequilibrium with the functional polymorphism. Such a functional polymorphism may alter splicing sites, affect the stability or transport of mRNA, or otherwise affect the transcription or translation of the nucleic acid. The presence of nucleotide sequence variants associated with functional polymorphism is diagnostic for susceptibility to AMI.

While we have genotyped and included a limited number of example SNP markers in the experimental section, any functional, regulatory or other mutation or alteration described above in any of the AMI risk genes identified herein is expected to predict the risk of AMI.

In diagnostic assays determination of the nucleotides present in one or several of the AMI associated SNP markers of this invention, as well as polymorphic sites associated with AMI associated SNP markers of this invention, in an individual's nucleic acid can be done by any method or technique which can accurately determine nucleotides present in a polymorphic site. Numerous suitable methods have been described in the art (Kwok P-Y, 2001; Syvänen A-C, 2001), these methods include, but are not limited to, hybridization assays, ligation assays, primer extension assays, enzymatic cleavage assays, chemical cleavage assays and any combinations of these assays. The assays may or may not include PCR, solid phase step, modified oligonucleotides, labeled probes or labeled nucleotides and the assay may be multiplex or singleplex. As it is obvious in the art the nucleotides present in polymorphic site can be determined from one nucleic acid strand or from both strands.

In another embodiment of the invention, diagnosis of a susceptibility to AMI can also be made by examining transcription of one or several AMI associated genes. Alterations in transcription can be analysed by a variety of methods as described in the art, including e.g. hybridization methods, enzymatic cleavage assays, RT-PCR assays and microarrays. A test sample from an individual is collected and the alterations in the transcription of AMI associated genes are assessed from the RNA present in the sample. Altered transcription is diagnostic for a susceptibility to AMI.

In another embodiment of the invention, diagnosis of a susceptibility to AMI can also be made by examining expression and/or structure and/or function of an AMI susceptibility polypeptide. A test sample from an individual is assessed for the presence of an alteration in the expression and/or an alteration in structure and/or function of the polypeptide encoded by an AMI risk gene, or for the presence of a particular polypeptide variant (e.g., an isoform) encoded by an AMI risk gene. An alteration in expression of a polypeptide encoded by an AMI risk gene can be, for example, an alteration in the quantitative polypeptide expression (i.e., the amount of polypeptide produced); an alteration in the structure and/or function of a polypeptide encoded by an AMI risk gene is an alteration in the qualitative polypeptide expression (e.g., expression of a mutant AMI susceptibility polypeptide or of a different splicing variant or isoform). In a preferred embodiment, detecting a particular splicing variant encoded by an AMI risk gene, or a particular pattern of splicing variants makes diagnosis of the disease or condition associated with AMI or a susceptibility to a disease or condition associated with AMI.

Alterations in expression and/or structure and/or function of an AMI susceptibility polypeptide can be determined by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition of polypeptide by an AMI risk gene in a control sample. A control sample is a sample that corresponds to the test sample (e.g., is from the same type of cells), and is from an individual who is not affected by AMI. An alteration in the expression or composition of the polypeptide in the test sample, as compared with the control sample, is indicative of a susceptibility to AMI.

Western blotting analysis, using an antibody as described above that specifically binds to a polypeptide encoded by a mutant AMI risk gene, or an antibody that specifically binds to a polypeptide encoded by a non-mutant gene, or an antibody that specifically binds to a particular splicing variant encoded by an AMI risk gene, can be used to identify the presence in a test sample of a particular splicing variant or isoform, or of a polypeptide encoded by a polymorphic or mutant AMI risk gene, or the absence in a test sample of a particular splicing variant or isoform, or of a polypeptide encoded by a non-polymorphic or non-mutant gene. The presence of a polypeptide encoded by a polymorphic or mutant gene, or the absence of a polypeptide encoded by a non-polymorphic or non-mutant gene, is diagnostic for a susceptibility to AMI, as is the presence (or absence) of particular splicing variants encoded by an AMI risk gene.

In one embodiment of this method, the level or amount of polypeptide encoded by an AMI risk gene in a test sample is compared with the level or amount of the polypeptide encoded by an AMI risk gene in a control sample. A level or amount of the polypeptide in the test sample that is higher or lower than the level or amount of the polypeptide in the control sample, such that the difference is statistically significant, is indicative of an alteration in the expression of the polypeptide encoded by an AMI risk gene, and is diagnostic for a susceptibility to AMI. Alternatively, the composition of the polypeptide encoded by an AMI risk gene in a test sample is compared with the composition of the polypeptide encoded by an AMI risk gene in a control sample (e.g., the presence of different splicing variants). A difference in the composition of the polypeptide in the test sample, as compared with the composition of the polypeptide in the control sample, is diagnostic for a susceptibility to AMI. In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a susceptibility to AMI.

In another embodiment, assessment of the splicing variant or isoform(s) of a polypeptide encoded by a polymorphic or mutant AMI risk gene can be performed. The assessment can be performed directly (e.g., by examining the polypeptide itself), or indirectly (e.g., by examining the mRNA encoding the polypeptide, such as through mRNA profiling). For example, probes or primers as described herein can be used to determine which splicing variants or isoforms are encoded by an AMI risk gene mRNA, using standard methods.

The presence in a test sample of a particular splicing variant(s) or isoform(s) associated with AMI or risk of AMI, or the absence in a test sample of a particular splicing variant(s) or isoform(s) not associated with AMI or risk of AMI, is diagnostic for a disease or condition associated with an AMI risk gene or a susceptibility to a disease or condition associated with an AMI risk gene. Similarly, the absence in a test sample of a particular splicing variant(s) or isoform(s) associated with AMI or risk of AMI, or the presence in a test sample of a particular splicing variant(s) or isoform(s) not associated with AMI or risk of AMI, is diagnostic for the absence of disease or condition associated with an AMI risk gene or a susceptibility to a disease or condition associated with an AMI risk gene.

The invention further pertains to a method for the diagnosis and identification of susceptibility to AMI in an individual, by identifying an at-risk allele or an at-risk haplotype in an AMI risk gene. In one embodiment, the at-risk allele or the at-risk haplotype is an allele or a haplotype for which the presence of the haplotype increases the risk of AMI significantly. Although it is to be understood that identifying whether a risk is significant may depend on a variety of factors, including the specific disease, the haplotype, and often, environmental factors, the significance may be measured by an odds ratio or a percentage. In a further embodiment, the significance is measured by a percentage. In one embodiment, a significant risk is measured as an odds ratio of 0.8 or less or at least about 1.2, including by not limited to: 0.1, 0.2, 0.3, 0.4, 0.5, 0.6, 0.7, 0.8, 0.9, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, an odds ratio of at least 1.2 is significant. In a further embodiment, an odds ratio of at least about 1.5 is significant. In a further embodiment, a significant increase or decrease in risk is at least about 1.7. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase or reduction in risk is at least about 50%. It is understood however, that identifying whether a risk is medically significant may also depend on a variety of factors, including the specific disease, the allele or the haplotype, and often, environmental factors.

The invention also pertains to methods of diagnosing AMI or a susceptibility to AMI in an individual, comprising screening for an at-risk haplotype in the AMI risk gene that is more frequently present in an individual susceptible to AMI (affected), compared to the frequency of its presence in a healthy individual (control), wherein the presence of the haplotype is indicative of AMI or susceptibility to AMI. See tables 4, 5, 6, 7, 8, 10 and 11 for SNP markers that comprise haplotypes that can be used as screening tools. SNP markers from these lists represent at-risk haplotypes and can be used to design diagnostic tests for determining a susceptibility to AMI.

Kits (e.g., reagent kits) useful in the methods of diagnosis comprise components useful in any of the methods described herein, including for example, PCR primers, hybridization probes or primers as described herein (e.g., labeled probes or primers), reagents for genotyping SNP markers, reagents for detection of labeled molecules, restriction enzymes (e.g., for RFLP analysis), allele-specific oligonucleotides, DNA polymerases, RNA polymerases, marker enzymes, antibodies which bind to altered or to non-altered (native) AMI susceptibility polypeptide, means for amplification of nucleic acids comprising one or several AMI risk genes, or means for analyzing the nucleic acid sequence of one or several AMI risk genes or for analyzing the amino acid sequence of one or several AMI susceptibility polypeptides, etc. In one embodiment, a kit for diagnosing susceptibility to AMI can comprise primers for nucleic acid amplification of a region in an AMI risk gene comprising an at-risk haplotype that is more frequently present in an individual susceptible to AMI. The primers can be designed using portions of the nucleic acids flanking SNPs that are indicative of AMI.

This invention is based on the principle that one or a small number of genotypings are performed, and the mutations to be typed are selected on the basis of their ability to predict AMI and/or CHD. For this reason any method to genotype mutations in a genomic DNA sample can be used. If non-parallel methods such as real-time PCR are used, the typings are done in a row. The PCR reactions may be multiplexed or carried out separately in a row or in parallel aliquots.

Thus, the detection method of the invention may further comprise a step of combining information concerning age, gender, the family history of hypertension, diabetes and hypercholesterolemia, and the medical history concerning CVD or diabetes of the subject with the results obtained from step b) of the method (see claim 1) for confirming the indication obtained from the detection step. Said information may also concern hypercholesterolemia in the family, smoking status, CHD in the family, history of CVD, obesity in the family, and waist-to-hip circumference ratio (cm/cm)

The detection method of the invention may also further comprise a step determining blood, serum or plasma cholesterol, HDL cholesterol, LDL cholesterol, triglyceride, apolipoprotein B and Al, fibrinogen, ferritin, transferrin receptor, C-reactive protein, serum or plasma insulin concentration.

The score that predicts the probability of AMI or CHD may be calculated using a multivariate failure time model or a logistic regression equation. The results from the furher steps of the method as described above render possible a step of calculating the probability of an AMI using a logistic regression equation as follows.

Probability of an AMI=1/[1+e (−(−a+Σ(bi*Xi))], where e is Napier's constant, Xi are variables related to the AMI, bi are coefficients of these variables in the logistic function, and a is the constant term in the logistic function, and wherein a and bi are preferably determined in the population in which the method is to be used, and Xi are preferably selected among the variables that have been measured in the population in which the method is to be used. Preferable values for bi are between −20 and 20; and for i between 0 (none) and 100,000. A negative coefficient bi implies that the marker is risk-reducing and a positive that the marker is risk-increasing.

Xi are binary variables that can have values or are coded as 0 (zero) or 1 (one) such as SNP markers. The model may additionally include any interaction (product) or terms of any variables Xi, e.g. biXi. An algorithm is developed for combining the information to yield a simple prediction of AMI as percentage of risk in one year, two years, five years, 10 years or 20 years.

Alternative statistical models are failure-time models such as the Cox's proportional hazards' model, other iterative models and neural networking models.

The test can be applied to test the risk of developing an AMI in both healthy persons, as a screening or predisposition test and high-risk persons (who have e.g. family history of CHD or elevated serum cholesterol or hypertension or diabetes or any combination of these or elevated level of any other coronary risk factor).

The method can be used in the prediction and early diagnosis of AMI in adult persons, stratification and selection of subjects in clinical trials, stratification and selection of persons for intensified preventive and curative interventions. The aim is to reduce the cost of clinical drug trials and health care.

Pharmaceutical Compositions

The present invention also pertains to pharmaceutical compositions comprising agents described herein, particularly nucleotides in AMI risk genes, and/or comprising other splicing variants encoded by AMI risk genes; and/or an agent that alters (e.g., enhances or inhibits) AMI risk genes expression or AMI susceptibility gene polypeptide activity as described herein. For instance, a polypeptide, protein (e.g., a receptor), an agent that alters an AMI risk gene expression, or an AMI susceptibility polypetide binding agent or binding partner, fragment, fusion protein or prodrug thereof, or a nucleotide or nucleic acid construct (vector) comprising a nucleotide of the present invention, or an agent that alters AMI susceptibility gene polypeptide activity, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of CHD, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Methods of Therapy

The present invention encompasses methods of treatment (prophylactic and/or therapeutic) for CHD, AMI or a susceptibility to AMI, such as individuals in the target populations described herein, using an AMI therapeutic agent. An “AMI therapeutic agent” is an agent that alters (e.g., enhances or inhibits) AMI risk affecting polypeptide (enzymatic activity or quantity) and/or an AMI risk gene expression, as described herein (e.g., an agonist or antagonist). AMI therapeutic agents can alter an AMI susceptibility polypeptide activity or nucleic acid expression by a variety of means, such as, for example, by providing additional AMI susceptibility polypeptide or by upregulating the transcription or translation of the AMI risk gene; by altering posttranslational processing of the AMI susceptibility polypeptide; by altering transcription of an AMI risk gene splicing variants; or by interfering with an AMI susceptibility polypeptide activity (e.g., by binding to an AMI susceptibility polypeptide); or by downregulating the transcription or translation of the AMI risk gene, or by inhibiting or enhancing the elimination of an AMI susceptibility polypeptide.

In particular, the invention relates to methods of treatment for AMI or susceptibility to AMI (for example, for individuals in an at-risk population such as those described herein); as well as to methods of treatment for manifestations and subtypes of CHD including but not limited to myocardial infarction, angina pectoris, atherosclerosis, acute coronary syndrome (e.g., unstable angina, non-ST-elevation myocardial infarction (NSTEMI) or ST-elevation myocardial infarction (STEMI)), peripheral arterial occlusive disease, cerebrovascular stroke, and complications or sequalae of AMI such as congestive heart failure and cardiac hypertrophy and arrythmias.

Representative AMI Therapeutic Agents Include the Following:

nucleic acids or fragments or derivatives thereof described herein, particularly nucleotides encoding the polypeptides described herein and vectors comprising such nucleic acids (e.g., a gene, cDNA, and/or mRNA, double-stranded interfering RNA, a nucleic acid encoding an AMI susceptibility polypeptide or active fragment or derivative thereof, or an oligonucleotide; for example, tables 3 through 11;

other polypeptides (e.g., AMI susceptibility receptors); AMI susceptibility polypeptide binding agents; peptidomimetics; fusion proteins or prodrugs thereof, antibodies (e.g., an antibody to a mutant AMI susceptibility polypeptide, or an antibody to a non-mutant AMI susceptibility polypeptide, or an antibody to a particular splicing variant encoded by an AMI risk gene, as described above); ribozymes; other small molecules;

and other agents that alter (e.g., inhibit or antagonize) an AMI risk gene expression or polypeptide activity, or that regulate transcription of an AMI risk gene splicing variants (e.g., agents that affect which splicing variants are expressed, or that affect the amount of each splicing variant that is expressed);

and other reagents that alter (e.g. induce or agonize) an AMI risk gene expression or polypeptide activity, or that regulate transcription of an AMI risk gene splicing variants (e.g., agents that affect which splicing variants are expressed, or that affect the amount of each splicing variant that is expressed).

More than one AMI therapeutic agent can be used concurrently, if desired.

The AMI therapeutic agent that is a nucleic acid is used in the treatment of AMI. The term, “treatment” as used herein, refers not only to ameliorating symptoms associated with the disease, but also preventing or delaying the onset of the disease, and also lessening the severity or frequency of symptoms of the disease, preventing or delaying the occurrence of a second episode of the disease or condition; and/or also lessening the severity or frequency of symptoms of the disease or condition. In the case of atherosclerosis, “treatment” also refers to a minimization or reversal of the development of plaques. The therapy is designed to alter (e.g., inhibit or enhance), replace or supplement activity of an AMI polypeptide in an individual. For example, an AMI therapeutic agent can be administered in order to upregulate or increase the expression or availability of an AMI risk gene or of specific splicing variants of an AMI susceptibility, gene or, conversely, to downregulate or decrease the expression or availability of an AMI risk gene or specific splicing variants of an AMI risk gene. Upregulation or increasing expression or availability of a native AMI risk gene or of a particular splicing variant could interfere with or compensate for the expression or activity of a defective gene or another splicing variant; downregulation or decreasing expression or availability of a native AMI risk gene or of a particular splicing variant could minimize the expression or activity of a defective gene or the particular splicing variant and thereby minimize the impact of the defective gene or the particular splicing variant.

The AMI therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding an AMI susceptibility polypeptide, such as tables 3 through 11 which may optionally comprise at least one polymorphism shown in tables 3 through 11; or another nucleic acid that encodes an AMI susceptibility polypeptide or a splicing variant, derivative or fragment thereof, can be used, either alone or in a pharmaceutical composition as described above. For example, an AMI risk gene or a cDNA encoding an AMI susceptibility polypeptide, either by itself or included within a vector, can be introduced into cells (either in vitro or in vivo) such that the cells produce native AMI susceptibility polypeptide. If necessary, cells that have been transformed with the gene or cDNA or a vector comprising the gene or cDNA can be introduced (or re-introduced) into an individual affected with the disease. Thus, cells which, in nature, lack of a native AMI risk gene expression and activity, or have mutant AMI risk gene expression and activity, or have expression of a disease-associated AMI risk gene splicing variant, can be engineered to express an AMI susceptibility polypeptide or an active fragment of an AMI susceptibility polypeptide (or a different variant of an AMI susceptibility polypeptide). In a preferred embodiment, nucleic acid encoding an AMI susceptibility polypeptide, or an active fragment or derivative thereof, can be introduced into an expression vector, such as a viral vector, and the vector can be introduced into appropriate cells in an animal. Other gene transfer systems, including viral and nonviral transfer systems, can be used. Alternatively, nonviral gene transfer methods, such as calcium phosphate coprecipitation, mechanical techniques (e.g., microinjection); membrane fusion-mediated transfer via liposomes; or direct DNA uptake, can also be used.

Alternatively, in another embodiment of the invention, a nucleic acid of the invention; a nucleic acid complementary to a nucleic acid of the invention; or a portion of such a nucleic acid (e.g., an oligonucleotide as described below), can be used in “antisense” therapy, in which a nucleic acid (e.g., an oligonucleotide) which specifically hybridizes to the mRNA and/or genomic DNA of an AMI risk gene is administered or generated in situ. The antisense nucleic acid that specifically hybridizes to the mRNA and/or DNA inhibits expression of the AMI susceptibility polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct of the present invention can be delivered, for example, as an expression plasmid as described above. When the plasmid is transcribed in the cell, it produces RNA which is complementary to a portion of the mRNA and/or DNA which encodes an AMI susceptibility polypeptide. Alternatively, the antisense construct can be an oligonucleotide probe which is generated ex vivo and introduced into cells; it then inhibits expression by hybridizing with the mRNA and/or genomic DNA of an AMI risk gene. In one embodiment, the oligonucleotide probes are modified oligonucleotides which are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA. Additionally, general approaches to constructing oligomers useful in antisense therapy are also described, for example, by van der Krol A R et al, 1988 and Stein C A and Cohen J S, 1988. With respect to antisense DNA, oligodeoxyribonucleotides derived from the translation initiation site, e.g., between the −10 and +10 regions of an AMI risk gene sequence, are preferred.

To perform antisense therapy, oligonucleotides (mRNA, cDNA or DNA) are designed that are complementary to mRNA encoding an AMI susceptibility polypeptide. The antisense oligonucleotides bind to AMI susceptibility mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g., for targeting host cell receptors in vivo), or agents facilitating transport across the cell membrane (Letsinger R L et al, 1989; Lemaitre M et al, 1987) or the blood-brain barrier (Jaeger L B and Banks W A, 2004), or hybridization-triggered cleavage agents (van der Krol A R et al, 1988) or intercalating agents. (Zon G, 1988). To this end, the oligonucleotide may be conjugated to another molecule (e.g., a peptide, hybridization triggered cross-linking agent, transport agent, hybridization-triggered cleavage agent).

The antisense molecules are delivered to cells that express an AMI risk gene in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a preferred embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous AMI risk gene transcripts and thereby prevent translation of the AMI susceptibility mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systemically).

An endogenous AMI risk gene expression can be also reduced by inactivating or “knocking out” an AMI risk gene or its promoter using targeted homologous recombination (Smithies O et al, 1985; Thomas K R and Capecchi M R, 1987; Thompson S et al, 1989). For example, a mutant, non-functional AMI risk gene (or a completely unrelated DNA sequence) flanked by DNA homologous to the endogenous AMI risk gene (either the coding regions or regulatory regions of an AMI risk gene) can be used, with or without a selectable marker and/or a negative selectable marker, to transfect cells that express an AMI risk gene in vivo. Insertion of the DNA construct, via targeted homologous recombination, results in inactivation of the AMI risk gene. The recombinant DNA constructs can be directly administered or targeted to the required site in vivo using appropriate vectors, as described above. Alternatively, expression of non-mutant AMI risk gene can be increased using a similar method: targeted homologous recombination can be used to insert a DNA construct comprising a non-mutant, functional AMI risk gene (e.g., any gene shown in tables 3 through 11 which may optionally comprise at least one polymorphism shown in tables 3 through 11), or a portion thereof, in place of a mutant AMI risk gene in the cell, as described above. In another embodiment, targeted homologous recombination can be used to insert a DNA construct comprising a nucleic acid that encodes an AMI susceptibility polypeptide variant that differs from that present in the cell.

Alternatively, an endogenous AMI risk gene expression can be reduced by targeting deoxyribonucleotide sequences complementary to the regulatory region of an AMI risk gene (i.e., the AMI risk gene promoter and/or enhancers) to form triple helical structures that prevent transcription of an AMI risk gene in target cells in the body (Helene C, 1991; Helene C et al, 1992; Maher L J, 1992). Likewise, the antisense constructs described herein, by antagonizing the normal biological activity of one of the AMI proteins, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to an AMI mRNA or gene sequence) can be used to investigate role of an AMI risk gene in developmental events, as well as the normal cellular function of an AMI risk gene in adult tissue. Such techniques can be utilized in cell culture, but can also be used in the creation of transgenic animals.

In yet another embodiment of the invention, other AMI therapeutic agents as described herein can also be used in the treatment or prevention of AMI. The therapeutic agents can be delivered in a composition, as described above, or by themshelves. They can be administered systemically, or can be targeted to a particular tissue. The therapeutic agents can be produced by a variety of means, including chemical synthesis; recombinant production; in vivo production, e.g. a transgenic animal (Meade H et al, 1990) and can be isolated using standard means such as those described herein.

A combination of any of the above methods of treatment (e.g., administration of non-mutant AMI susceptibility polypeptide in conjunction with antisense therapy targeting mutant AMI susceptibility mRNA; administration of a first splicing variant encoded by an AMI risk gene in conjunction with antisense therapy targeting a second splicing encoded by an AMI risk gene), can also be used.

This application includes sequence listing and tables that are submitted in electronic form. The sequence listing and tables are submitted herewith on one original and one duplicate compact disc (in compliance with 37 C.F.R. § 1.52(e)) designated respectively as Copy 1 Replacement and Copy 2 Replacement, and labeled in compliance with 37 C.F.R. § 1.52(e)(6). All the material in the sequence listing and tables on compact disc is hereby incorporated in their entirety herein by reference, and identified by the following data of file names, creation date and size in bytes:

| FILE NAME | CREATED | SIZE IN BYTES |
|---|---|---|
| 2005-09-07 0933- | 07-Sep-05 | 297 000 |
| 0245PUS1.ST25.txt | | |
| Table3.txt | 11-Aug-05 | 41 000 |
| Table4.txt | 11-Aug-05 | 76 000 |
| Table5.txt | 11-Aug-05 | 81 000 |
| Table6.txt | 11-Aug-05 | 80 000 |
| Table7.txt | 11-Aug-05 | 11 000 |
| Table8.txt | 11-Aug-05 | 13 000 |
| Table9.txt | 11-Aug-05 | 59 000 |
| Table10.txt | 11-Aug-05 | 5 000 |
| Table11.txt | 11-Aug-05 | 5 000 |

The invention will be further described by the following non-limiting examples. The teachings of all publications cited herein are incorporated herein by reference in their entirety.

Experimental Section

East Finnish AMI Patients and Phenotype Characterization

The subjects were participants of the Kuopio Ischaemic Heart Disease Risk Factor Study (KIHD), which is an ongoing prospective population-based study designed to investigate risk factors for chronic diseases, including AMI and CVD, among middle-aged men (Salonen J T 1988, Salonen J T et al 1999, Tuomainen T-P et al 1999). The study population was a random age-stratified sample of men living in Eastern Finland who were 42, 48, 54 or 60 years old at baseline examinations in 1984-1989. A total of 2682 men were examined during 1984-89. The male cohort was complemented by a random population sample of 920 women, first examined during 1998-2001, at the time of the 11-year follow up of the male cohort. The follow-up of coronary events was to the end of 2002, providing a follow-up time ranging from 13 years to 18 years. The recruitment and examination of the subjects has been described previously in detail (Salonen J T 1988). The University of Kuopio Research Ethics Committee approved the study. All participants gave their written informed consent.

Data on CHD and AMI during the follow-up were obtained by computer record linkage to the national computerized hospital discharge registry. Diagnostic information was collected from the hospitals and all heart attacks events were classified according to rigid predefined criteria. The diagnostic classification of acute coronary events was based on symptoms, electrocardiographic findings, cardiac enzyme elevations, autopsy findings and the history of CHD. Each suspected coronary event (ICD-9 codes 410-414 and ICD-10 codes I20-I25) was classified into 1) a definite AMI, 2) a probable AMI, 3) a typical acute chest pain episode of more than 20 minutes indicating CHD, 4) an ischemic cardiac arrest with successful resuscitation, 5) no acute coronary event or 6) an unclassifiable fatal case. The categories 1) to 3) were combined for the present analysis to denote AMI.

The cases were defined so that they had either a confirmed definite or probable AMI or typical prolonged chest pain and a family history of AMI (at least one affected family member, either a sibling or a parent). These characteristics were determined to increase the likelihood that the coronary disease in the case subjects was caused by genes and not by non-genetic factors. Analogically, the controls did not have family history of AMI in either their parents of siblings.

An identical number of healthy control subjects were selected from the same KIHD cohort as the cases. They had no family history of CHD in parents or siblings. To minimize the control-dilution bias (controls developing AMI later), CHD-free controls were selected from very healthy persons. The controls were free of CHD, assessed broadly. The controls for GWS had neither diagnosed CHD, symptoms or signs of CHD, nitroglycerin medication, ischaemic ECG findings in maximal exercise test, type 2 diabetes nor moderate-to-severe hypertension. The proportion of males was equal among both the cases and the controls. To control for confounding, the controls were matched according to gender, smoking status and the municipality of residence. In this founder-population-based familial case-control design, the number of both the cases and the controls used in the initial GWS was 125 (125+125=250). Selected characteristics of the cases and controls are shown in table 2. As the controls had been matched, the age and the number of cigarettes smoked daily were similar in both groups.

TABLE 2
Selected characteristics of the cases and controls

| | Cases (n = 125) | | | Controls (n = 125) | | |
|---|---|---|---|---|---|---|
| | Mean | Min | Max | Mean | Min | Max |
| Age (years) | 54.5 | 42.1 | 71.9 | 54.2 | 42.3 | 71.8 |
| Cigarettes/day | 6.1 | 0 | 40 | 5.9 | 0 | 30 |
| S-Cholesterol (mmol/L) | 6.2 | 3.6 | 8.7 | 5.8 | 3.1 | 9.1 |
| S-HDL-Chol (mmol/L) | 1.25 | 0.80 | 2.77 | 1.35 | 0.76 | 2.38 |
| P-fibrinogen (g/L) | 3.16 | 1.77 | 4.80 | 2.94 | 2.14 | 5.11 |
| B-Glucose (mmol/L) | 5.00 | 3.6 | 12.6 | 4.54 | 3.3 | 5.9 |
| S-Insulin (U/L) | 12.7 | 1.7 | 59.6 | 9.6 | 3.0 | 50.0 |

In table 2 are summarized selected characteristics of the cases and controls. Age and tobacco smoking were recorded on a self-administered questionnaire checked by an interviewer. Fasting blood glucose was measured using a glucose dehydrogenase method after precipitation of proteins by trichloroacetic acid. Serum insulin was determined with a Novo Biolabs radioimmunoassay kit (Novo Nordisk). HDL fractions were separated from fresh serum by combined ultracentrifugation and precipitation. The cholesterol contents of lipoprotein fractions and serum triglycerides were measured enzymatically. Fibrinogen was measured based on the clotting of diluted plasma with excess thrombin.

Hypertension was defined as either systolic blood pressure (SBP)≧2165 mmHg or diastolic BP (DBP)≧95 mmHg or antihypertensive treatment. Both blood pressures were measured in the morning by a nurse with a random-zero mercury sphygmomanometer. The measuring protocol included three measurements in supine, one in standing and two in sitting position with 5-minutes intervals. The mean of all six measurements were used as SBP and DBP.

Family history of CHD was defined positive if the subject's mother, father or a sibling had a history of either AMI or angina pectoris. Family histories of cerebrovascular stroke and diabetes were defined similarly. Adulthood socioeconomical status (SES) is an index comprised of measures of education, occupation, income and material living conditions. The scale is inverse, low score corresponding to high SES. These data have been collected by a self administered questionnaire.

Serum ferritin was assessed with a commercial double antibody radioimmunoassay (Amersham International, Amersham, UK). Lipoproteins, including high density lipoprotein (HDL) and low density lipoprotein (LDL), were separated from fresh serum samples by ultracentrifugation followed by direct very low density lipoprotein (VLDL) removal and LDL precipitation (Salonen et al 1991). Cholesterol concentration was then determined enzymically. Serum C-reactive protein was measured by a commercial high-sensitive immunometric assay (Immulite High Sensitivity CR Assay, DPC, Los Angeles).

Genomic DNA Isolation and Quality Testing

High molecular weight genomic DNA samples were extracted from frozen venous whole blood using standard methods and dissolved in standard TE buffer. The quantity and purity of each DNA sample was evaluated by measuring the absorbance at 260 and 280 nm and integrity of isolated DNA samples was evaluated with 0.9% agarose gel electrophoresis and Ethidiumbromide staining. A sample was qualified for genome wide scan (GWS) analysis if A260/A280 ratio was ≧1.7 and average size of isolated DNA was over 20 kb in agarose gel electrophoresis. Before GWS analysis samples were diluted to concentration of 50 ng/μl in reduced EDTA TE buffer (TEKnova).

Genome-Wide Scan

Genotyping of SNP markers was performed by using the technology access version of Affymetrix GeneChip® human mapping 100 k system. The assay consisted of two arrays, Xba and Hind, which were used to genotype over 126,000 SNP markers from each DNA sample. The assays were performed according to the instructions provided by the manufacturer. A total of 250 ng of genomic DNA was used for each individual assay. DNA sample was digested with either Xba I or Hind III enzyme (New England Biolabs, NEB) in the mixture of NE Buffer 2 (1×; NEB), bovine serum albumin (1×; NEB), and either Xba I or Hind III (0.5 U/μl; NEB) for 2h at +37° C. followed by enzyme inactivation for 20 min at +70° C. Xba I or Hind III adapters were then ligated to the digested DNA samples by adding Xba or Hind III adapter (0.25 μM, Affymetrix), T4 DNA ligase buffer (1×; NEB), and T4 DNA ligase (250 U; NEB). Ligation reactions were allowed to proceed for 2 h at +16° C. followed by 20 min incubation at +70° C. Each ligated DNA sample was diluted with 75 μl of molecular biology-grade water (BioWhittaker Molecular Applications/Cambrex).

Diluted ligated DNA samples were subjected to four identical 100 μl volume polymerase chain reactions (PCR) by implementing a 10 μl aliquot of DNA sample with Pfx Amplification Buffer (1×; Invitrogen), PCR Enhancer (1×; Invitrogen), MgSO₄ (1 mM; Invitrogen), dNTP (300 μM each; Takara), PCR primer (1 μM; Affymetrix), and Pfx Polymerase (0.05 U/μl; Invitrogen). The PCR was allowed to proceed for 3 min at +94° C., followed by 30 cycles of 15 sec at +94° C., 30 sec at +60° C., 60 sec at +68° C., and finally for the final extension for 7 min at +68° C. The performance of the PCR was checked by standard 2% agarose gel electrophoresis in 1× TBE buffer for 1 h at 120V.

PCR products were purified according to Affymetrix manual using MinElute 96 UF PCR Purification kit (Qiagen) by combining all four PCR products of an individual sample into same purification reaction. The purified PCR products were eluted with 40 μl of EB buffer (Qiagen), and the yields of the products were measured at the absorbance 260 nm. A total of 40 μg of each PCR product was then subjected to fragmentation reaction consisting of 0.2 U/μl fragmentation reagent (Affymetrix) in 1× Fragmentation Buffer. Fragmentation reaction was allowed to proceed for 35 min at +37° C. followed by 15 min incubation at +95° C. for enzyme inactivation. Completeness of fragmentation was checked by running an aliquot of each fragmented PCR product in 4% agarose 1× TBE (BMA Reliant precast) for 30-45 min at 120V.

Fragmented PCR products were then labeled using 1× Terminal Deoxinucleotidyl Transferase (TdT) buffer (Affymetrix), GeneChip DNA Labeling Reagent (0.214 mM; Affymetrix), and TdT (1.5 U/μl; Affymetrix) for 2 h at +37° C. followed by 15 min at +95° C. Labeled DNA samples were combined with hybridization buffer consisting of 0.056 M MES solution (Sigma), 5% DMSO (Sigma), 2.5× Denhardt's solution (Sigma), 5.77 mM EDTA (Ambion), 0.115 mg/ml Herring Sperm DNA (Promega), 1× Oligonucleotide Control reagent (Affymetrix), 11.5 μg/ml Human Cot-1 (Invitrogen), 0.0115% Tween-20 (Pierce), and 2.69 M Tetramethyl Ammonium Chloride (Sigma). DNA-hybridization buffer mix was denatured for 10 min at +95° C., cooled on ice for 10 sec and incubated for 2 min at +48° C. prior to hybridization onto corresponding Xba or Hind GeneChip® array. Hybridization was completed at +48° C. for 16-18 h at 60 rpm in an Affymetrix GeneChip Hybridization Oven. Following hybridization, the arrays were stained and washed in GeneChip Fluidics Station 450 according to fluidics station protocol Mapping10Kv1_{—}450 as recommended by the manufacturer. Arrays were scanned with GeneChip 3000 Scanner and the genotype calls for each of the SNP markers on the array were generated using Affymetrix Genotyping Tools (GTT) software. The confidence score in SNP calling algorithm was adjusted to 0.20.

Initial SNP Selection for Statistical Analysis

Prior to the statistical analysis, SNP quality was assessed on the basis of three values: the call rate (CR), minor allele frequency (MAF), and Hardy-Weinberg equilibrium (H-W). The CR is the proportion of samples with successful genotyping result. It does not take into account whether the genotypes are correct or not. The call rate was calculated as: CR=number of samples with successful genotype call/total number of samples. The MAF is the frequency of the allele that is less frequent in the study sample. MAF was calculated as: MAF=min(p, q), where p is frequency of the SNP allele ‘A’ and q is frequency of the SNP allele ‘B’; p=(number of samples with “AA”-genotype+0.5*number of samples with “AB”-genotype)/total number of samples with successful genotype call; q=1−p. SNPs that are homozygous (MAF=0) can not be used in genetic analysis and were thus discarded. H-W equilibrium is tested for controls. The test is based on the standard Chi-square test of goodness of fit. The observed genotype distribution is compared with the expected genotype distribution under H-W equilibrium. For two alleles this distribution is p², 2pq, and q² for genotypes ‘AA’, ‘AB’ and ‘BB’, respectively. If the SNP is not in H-W equilibrium it can be due to genotyping error or some unknown population dynamics (e.g. random drift, selection).

Only the SNPs that had CR>50%, MAF>1%, and were in H-W equilibrium (Chi-square test statistic<23.93) were used in the statistical analysis. A total of 107,895 SNPs fulfilled the above criteria and were included in the statistical analysis.

Statistical Methods

Single SNP Analysis

Differences in allele distributions between cases and controls were screened for all 107,895 SNPs. The screening was carried out by using the standard Chi-square independence test with 1 df (allele distribution, 2×2 table). SNPs that gave P-value less than 0.005 (Chi-square distribution with 1 df of 7.88 or more) were considered as statistically significant and selected for further analysis. There were 656 SNPs that fulfilled this criterium.

Haplotype Analysis

The data set was analyzed with a haplotype pattern mining algorithm either with HPM-G software (Sevon P et al, 2004) or with HPM software (Toivonen H T et al, 2000). For HPM software genotypes must have phase known i.e. to determine which alleles are coming from the mother and which from the father. Without family data phases must be estimated based on population data. We used HaploRec-program (Eronen L et al, 2004) to estimate the phases. HPM-G and HPM are very fast and can handle a large number of SNPs in a single run

The difference between HPM and HPM-G is that HPM-G can use phase unknown genotypic data and HPM uses phase known (or estimated by HaploRec or similar program) data. HPM-G finds all haplotype patterns that fit the genotype configuration. For phase-known data HPM finds all haplotype patterns that are in concordance with the phase configuration. The length of the haplotype patterns can vary. As an example, if there are four SNPs and an individual has alleles A T for the SNP1, C C for the SNP2, C G for the SNP3, and A C for the SNP4 then HPM-G considers haplotype patterns (of length 4 SNPs): ACCA, TCGC, TCCA, ACGC, ACGA, TCCC, TCGA, ACCC. HPM considers only haplotype patterns that are in concordance with estimated phase (done by HaploRec). If the estimated phase is ACGA (from the mother/father) and TCCC (from the father/mother) then HPM considers only two patterns (of length 4 SNPs): ACGA and TCCC.

A SNP is scored based on the number of times it is included in a haplotype pattern that differs between cases and controls (a threshold Chi-square value can be selected by the user). Significance of the score values is tested based on permutation tests.

Several parameters can be modified in the HPM-G and HPM programs including the Chi-square threshold value (−x), the maximum haplotype pattern length (−l), the maximum number of wildcards that can be included in a haplotype pattern (−w), and the number of permutation test in order to estimate the P-value (−p). Wildcards allow gaps in haplotypes. The HPM-G program was run with the following parameter settings: 1) haplotype analysis with 5 SNPs (−x9−15−w1−p10000) 2), haplotype analysis with 8 SNPs (−x9−18−w2−p10000). HPM was run with the following parameter settings: haplotype analysis with 5 SNPs (−x9−15−w1−p10000). Based on 10,000 replicates (−p10000) in the HPM-G analyses 1067 SNPs were significant at P-value less than 0.005 and 802 in the HPM analysis. According to both methods a total of 1665 SNPs were significant at P-value less than 0.005 (204 SNPs were selected by both analyses).

Definition of terms used in the haplotype analysis results.

The term “haplotype genomic region” or “haplotype region” refers to a genomic region that has been found significant in the haplotype analysis (HPM, HPMG or similar statistical method/program). The haplotype region is defined as 100 Kbp up/downstream from the physical position the first/last SNP that was included in the statistical analysis (haplotype analysis) and was found statistically significant. This region is given in based pairs based on the given genome build e.g. SNP physical position (basepair position) according to NCBI Human Genome Build 35.

The term “haplotype”, as described herein, refers to any combination of alleles e.g. T G C A that is found in the given genetic markers e.g. rs834485, rs856283, rs1260817, and rs1260772. A defined haplotype gives the name of the genetic markers (dbSNP rs-id for the SNPs) and the alleles. As it is recognized by those skilled in the art the same haplotype can be described differently by determining alleles from different strands e.g. the haplotype rs834485, rs856283, rs1260817, rs1260772 (T G C A) is the same as haplotype rs834485, rs856283, rs1260817, rs1260772 (A C G T) in which the alleles are determined from the other strand or haplotype rs834485, rs856283, rs1260817, rs1260772 (A G C A), in which the first allele is determined from the other strand.

The haplotypes described herein, e.g., having markers such as those shown in tables 4, 5, 6, 7, 8, 10 and 11, are found more frequently in individuals with AMI than in individuals without AMI. Therefore, these haplotypes have predictive value for detecting AMI or a susceptibility to AMI in an individual. Therefore, detecting haplotypes can be accomplished by methods known in the art for detecting sequences at polymorphic sites.

It is understood that the AMI associated at-risk alleles and at-risk haplotypes described in this invention may be associated with other “polymorphic sites” located in AMI associated genes of this invention. These other AMI associated polymorphic sites may be either equally useful as genetic markers or even more useful as causative variations explaining the observed association of at-risk alleles and at-risk haplotypes of this invention to AMI.

Multivariate Modeling

Of the 656 SNPs from the screening of individual markers and 1665 SNPs from the haplotype pattern analyses, there were 2039 SNPs (282 SNPs were the same in both screens). For modelling 1465 strongest predicting SNP markers were tested for entry. These were recoded as dummy variables in two ways: a) Homozygote of the minor allele coded as 1, otherwise 0, and b) Carrier of the minor allele coded as 1, otherwise 0. A multivariate binary logistic function regression analysis was used to: a) Find the SNPs that were most predictive of AMI and b) Construct a multivariate model that predicted AMI the strongest.

A forward step-up model construction was used with p-value to enter of 0.01 and p-value to exclude from the model of 0.02. The predictivity of the models was estimated by two methods: the Nagelkerke R square and the reclassification of the subjects to cases and controls on the basis of the logistic model contructed. The predicted probability used as cut-off was 0.5. A data reduction analysis was carried out by step-down and step-up logistic modeling. The statistical software used was SPSS for Windows, version 11.5.

Results

In table 3 (on CD) are summarized the characteristics of the SNP markers with the strongest association with AMI in the individual marker analysis. SNP identification number according to NCBI dbSNP database build 124. SNP physical position according to NCBI Human Genome Build 35. Gene locus as reported by NCBI dbSNP database build 124. SNP flanking sequence provided by Affymetrix “csv” commercial access Human Mapping 100K array annotation files.

In table 4 (on CD) are summarized the characteristics of the haplotype genomic regions with the strongest association with AMI in the HPM-G analysis with 5 SNPs. SNP identification number according to NCBI dbSNP database build 124. SNP physical position according to NCBI Human Genome Build 35. Associated genes are those genes positioned within 100 Kbp up/downstream from the physical position of the SNPs bordering the haplotype genomic region found using NCBI MapViewer, based on NCBI Human Genome Build 35. SNP flanking sequence provided by Affymetrix “csv” commercial access Human Mapping 100K array annotation files.

In table 5 (on CD) are summarized the characteristics of the haplotype genomic regions with the strongest association with AMI in the HPM-G analysis with 8 SNPs. SNP identification number according to NCBI dbSNP database build 124. SNP physical position according to NCBI Human Genome Build 35. Associated genes are those genes positioned within 100 Kbp up/downstream from the physical position of the SNPs bordering the haplotype genomic region found using NCBI MapViewer, based on NCBI Human Genome Build 35. SNP flanking sequence provided by Affymetrix “csv” commercial access Human Mapping 100K array annotation files.

In table 6 (on CD) are summarized the characteristics of the haplotype genomic regions with the strongest association with AMI in the HPM analysis with 8 SNPs. SNP identification number according to NCBI dbSNP database build 124. SNP physical position according to NCBI Human Genome Build 35. Associated genes are those genes positioned within 100 Kbp up/downstream from the physical position of the SNPs bordering the haplotype genomic region found using NCBI MapViewer, based on NCBI Human Genome Build 35. SNP flanking sequence provided by Affymetrix “csv” commercial access Human Mapping 100K array annotation files.

In table 7 (on CD) are listed haplotype blocks with the strongest association with AMI based on HPM-G analysis. SNP identification number according to NCBI dbSNP database build 124.

In table 8 (on CD) are listed haplotype blocks with the strongest association with AMI based on HaploRec +HPM analysis. SNP identification number according to NCBI dbSNP database build 124.

In table 9 (on CD) are listed all genes found associated with AMI according to point wise or haplotype analyses. Gene name according to HUGO Gene Nomenclature Committee (HGNC).

In table 10 (on CD) are listed the SNP-markers and haplotypes that best predicted risk of AMI in a multivariate logistic model. The model was constructed by a step-up procedure, using P=0.01 for entry criterium and P=0.02 as exclusion criterium. For modelling, 1465 strongest predicting SNP markers and 27 haplotypes were tested for entry. SNP identification number according to NCBI dbSNP database build 124. The models includes five haplotypes and seven individual SNP markers. The 12-variable model predicts 95.2% of AMIs correctly. The statistics are based on 125 KIHD participants who developed AMI during 1984 to 2002 and 125 KIHD participants who neither had any CHD at KIHD baseline and who remained free of clinical AMI during the follow-up up the end of 2002. The controls were matched according to gender, age, place of residence and smoking status.

The table 11 (on CD) shows another example of a multivariate logistic model, in which the haplotypes were contructed after selecting the strongest predicting genomic regions as described above. The model was contructed identically. In addition to five haplotypes and seven SNP markers, also two phenotypic variables were entered. The model predicted 95.6% of AMIs.

Implications and Conclusions

We have found associations between 2039 SNP markers and the risk of AMI in a population-based prospective nested set of familial cases and extremely healthy controls. Of these, 656 were identified in the analysis of individual SNPs and 1665 in haplotype sharing analysis. Of the 2039 markers, 283 predicted AMI in both types of statistical analysis. We further identified several sets of SNP markers and haplotypes, which predict in a multivariate logistic model virtually fully the development of AMI.

The results of the pointwise and haplotype analyses identified a total of 978 genes associated with AMI, of which 380 genes had at least one of the 2039 SNP markers physically linked to the gene.

The conventional wisdom since the 19^{th} century has been that AMI is caused by myocardial ischaemia, which is caused by reduction of blood flow in the coronary arteries. The blood flow, in turn, is reduced because of either constant arterial narrowing due to atherosclerosis, arterial occlusion or thrombus, or because of temporary arterial constriction, or by a combination of any two or three of these. However, a large proportion of the patients who have died because of an AMI, have no detectable arterial narrowing in autopsy. This suggests that also other pathways than coronary arterial narrowing must be able to cause AMI. We have identified four principal pathways in the etiology of AMI:
(1) Coronary arterial narrowing
   a. Due to coronary arteriosclerosis or atherosclerosis (caused by either dyslipoproteinemias, glucose and insulin metabolism disturbances, hypertension, platelet hyperactivity, enhanced coagulation, decreased fibrinolysis, inflammation or exaggerated immune response, disturbances in antioxidative or pro-oxidative systems, dysfunctional arterial damage repair or healing), or
   b. Due to coronary arterial thrombus, or
   c. Due to coronary arterial vasoconstriction,
(2) Cardiac arrhythmias or conduction disturbances,
(3) Myocardial phenomena and processes (other than ischaemia), and
(4) Embryologic and infantile development and growth of the heart and arteries.

As part of this invention, we were also able to identify more specific pathways through which the genes influence the risk of AMI. The pathways were: myocardial effects and processes; arrhythmias and cardiac conduction defects; inflammatory processes and immune response; lipid synthesis, absorption, distribution, metabolism, elimination and transport; platelet function, coagulation and fibrinolysis; glucose and insulin metabolism; blood pressuure regulation and hypertension; endogenous antioxidative and pro-oxidative ssytems; myocardial and arterial apoptosis; arterial effects and processes, atherosclerosis and arteriosclerosis; iron accumulation and metabolism; embryonic and infantile development and growth; transmembrane transport in heart and arteries; cell signalling in heart and arteries.

The number of genes discovered that act through direct myocardial effects and processes as well as during embryonic development and growth very surprisingly high. On the basis of our findings, the role of these pathways in the causation of CHD and AMI has been underestimated.

The emphasized role of myocardial phenomena in the etiology of CHD and AMI explains the previous observation, according to which a large proportion of patient with AMI do not have either severe coronary atherosclerosis or coronary thrombosis. It is plausible that an AMI may result from processes which are initiated in the myocardium itself, rather than as a consequence of ischemia, caused by reduced blood flow to myocardium, as conventionally thought. Also, direct myocardial effects may aggravate myocardial damage caused initially by ischemia due to reduced blood flow (Tun A and Khan I A, 2001; Waller B F et al, 1996; Virmani R et al, 2001; Franz W M et al, 2001; Gomes A V and Potter J D, 2004; Fatkin D and Graham R M, 2002).

The second pathway to AMI which appears to be more important than earlier thought, is the embryonic and infantile development and growth. It is plausible that the early development influences the vulnerability of both myocardium and arteries to injury and damage, and possibly also the defensive and repair systems in both of these. There is no prior evidence supporting our findings, most likely do to the difficulty of studying this subject in humans.

Thus, we have discovered a total of 978 AMI genes, in which any genetic markers can be used to predict AMI, and thus these markers can be used as part of molecular diagnostic tests of AMI predisposition. In addition, we have disclosed a set of 2039 SNP markers which are predictive of AMI. The markers can also be used as part of pharmacogenetic tests which predict the efficacy and adverse reactions of anti-coronary agents and compounds. The genes discovered are also targets to new therapies of AMI, such as drugs. Other therapies are molecular, including gene transfer. The new genes can also be used to develop and produce new transgenic animals for studies of anti-coronary agents and compounds.

While this invention has been particularly shown and described with reference to preferred embodiments thereof, it will be understood by those skilled in the art that various changes in form and details may be made therein without departing from the spirit and scope of the invention as defined by the appended claims.

### REFERENCES

American Heart Association. Heart Disease and Stroke Statistics—2004 Update. http://www.americanheart.org/downloadable/heart/1079736729696HDSStats2004UpdateRE V3-19-04.pdf (Accessed 14.07.04).
Ardissino D, Mannucci P M, Merlini P A, Duca F, Fetiveau R, Tagliabue L, Tubaro M, Galvani M, Ottani F, Ferrario M, Corral J, Margaglione M. 1999. Prothrombotic genetic risk factors in young survivors of myocardial infarction. Blood. 94:46-51.
Ausubel F M, Brent R, Kingston R E, Moore D D, Seidman J G, Struhl K. 2003. Current Protocols in Molecular Biology. NY:John Wiley & Sons.
Baroni M G, Berni A, Romeo S, Arca M, Tesorio T, Sorropago G, Di Mario U, Galton D J. 2003. Genetic study of common variants at the Apo E, Apo AI, Apo CIII, Apo B, lipoprotein lipase (LPL) and hepatic lipase (LIPC) genes and coronary artery disease (CAD): variation in LIPC gene associates with clinical outcomes in patients with established CAD. BMC Med Genet. 4:8.
Barrett-Connor E, Khaw K. 1984. Family history of heart attack as an independent predictor of death due to cardiovascular disease. Circulation 69: 1065-9.
Beyzade S, Zhang S, Wong Y K, Day I N, Eriksson P, Ye S. 2003. Influences of matrix metalloproteinase-3 gene variation on extent of coronary atherosclerosis and risk of myocardial infarction. J Am Coll Cardiol. 41:2130-7.
Bierer B, Coligan J E, Margulies D H, Shevach E M, Strober W. 2002. Current Protocols in Immunology. NY:John Wiley & Sons.
Boer J M, Feskens E J, Verschuren W M, Seidell J C, Kromhout D. 1999. The joint impact of family history of myocardial infarction and other risk factors on 12-year coronary heart disease mortality. Epidemiology 10:767-70.
Broeckel U, Hengstenberg C, Mayer B, Holmer S, Martin L J, Comuzzie A G, Blangero J, Nurnberg P, Reis A, Riegger G A, Jacob H J, Schunkert H. 2002. A comprehensive linkage analysis for myocardial infarction and its related risk factors. Nat Genet. 30:210-4.
Brscic E, Bergerone S, Gagnor A, Colajanni E, Matullo G, Scaglione L, Cassader M, Gaschino G, Di Leo M, Brusca A, Pagano G F, Piazza A, Trevi G P. 2000. Acute myocardial infarction in young adults: prognostic role of angiotensin-converting enzyme, angiotensin II type I receptor, apolipoprotein E, endothelial constitutive nitric oxide synthase, and glycoprotein IIIa genetic polymorphisms at medium-term follow-up. Am Heart J. 139:979-84.
Burzotta F, Paciaroni K, De Stefano V, Crea F, Maseri A, Leone G, Andreotti F. 2004. G20210A prothrombin gene polymorphism and coronary ischaemic syndromes: a phenotype-specific meta-analysis of 12 034 subjects. Heart. 90:82-6.
Chao T H, Li Y H, Chen J H, Wu H L, Shi G Y, Tsai W C, Chen P S, Liu P Y. 2004. Relation of thrombomodulin gene polymorphisms to acute myocardial infarction in patients < or =50 years of age. Am J Cardiol. 93:204-7.
Chen X, Levine L, Kwok P Y. 1999. Fluorescence polarization in homogeneous nucleic acid analysis. Genome Res. 9:492-8.
Chiodini B D, Barlera S, Franzosi M G, Beceiro V L, Introna M, Tognoni G. 2003. APO B gene polymorphisms and coronary artery disease: a meta-analysis. Atherosclerosis. 167:355-66.
Chiodini B D, Lewis C M. 2003. Meta-analysis of 4 coronary heart disease genome-wide linkage studies confirms a susceptibility locus on chromosome 3q. Arterioscler Thromb Vasc Biol. 23:1863-8.
Chisolm G M, Steinberg D. 2000. The oxidative modification hypothesis of atherogenesis: an overview. Free Radic Biol Med 28: 1815-26.
Church G M, Gilbert W. 1984. Genomic sequencing. Proc Natl Acad Sci U S A. 81:1991-5.
Cine N, Hatemi A C, Erginel-Unaltuna N. 2002. Association of a polymorphism of the ecNOS gene with myocardial infarction in a subgroup of Turkish MI patients. Clin Genet. 61:66-70.
Civeira F. 2004. International Panel on Management of Familial Hypercholesterolemia. Guidelines for the diagnosis and management of heterozygous familial hypercholesterolemia. Atherosclerosis. 173:55-68.
Colditz G A, Rimm E B, Giovannucci E, Stampfer M J, Rosner B, Willett W C. 1991. A prospective study of parental history of myocardial infarction and coronary artery disease in men. Am J Cardiol 67: 933-8.
Colditz G A, Stampfer M J, Willett W C, Rosner B, Speizer F E, Hennekens C H. 1986. A prospective study of parental history of myocardial infarction and coronary heart disease in women. Am J Epidemiol 123: 48-58.
Cole S P, Campling B G, Atlaw T, Kozbor D, Roder J C. 1984. Human monoclonal antibodies. Mol Cell Biochem. 62:109-20.
Davies M J. 1990. A macro and micro view of coronary vascular insult in ischemic heart disease. Circulation 82(3 Suppl): 1138-46.
Deiman B, van Aarle P, Sillekens P. 2002. Characteristics and applications of nucleic acid sequence-based amplification (NASBA). Mol Biotechnol. 20:163-79.
Dianzani I, Forrest S M, Camaschella C, Gottardi E, Cotton R G. 1991. Heterozygotes and homozygotes: discrimination by chemical cleavage of mismatch. Am J Hum Genet. 48:423-4.
Eckert K A, Kunkel T A. 1991. DNA polymerase fidelity and the polymerase chain reaction. PCR Methods Appl. 1:17-24.
Eichner J E, Kuller L H, Orchard T J, Grandits G A, McCallum L M, Ferrell R E, Neaton J D. 1993. Relation of apolipoprotein E phenotype to myocardial infarction and mortality from coronary artery disease. Am J Cardiol. 71:160-5.
Eronen L, Geerts F, Toivonen H. 2004. A Markov chain approach to reconstruction of long haplotypes. Pac Symp Biocomput.:104-15.
Fatkin D, Graham R M. 2002. Molecular mechanisms of inherited cardiomyopathies. Physiol Rev. 82:945-80.
Fodor S P, Read J L, Pirrung M C, Stryer L, Lu A T, Solas D. 1991. Light-directed, spatially addressable parallel chemical synthesis. Science. 251:767-73.
Fox C S, Cupples L A, Chazaro I, Polak J F, Wolf P A, D'Agostino R B, Ordovas J M, O'Donnell C J. 2004. Genomewide linkage analysis for internal carotid artery intimal medial thickness: evidence for linkage to chromosome 12. Am J Hum Genet. 74:253-61.
Francke S, Manraj M, Lacquemant C, Lecoeur C, Lepretre F, Passa P, Hebe A, Corset L, Yan S L, Lahmidi S, Jankee S, Gunness T K, Ramjuttun U S, Balgobin V, Dina C, Froguel P. 2001. A genome-wide scan for coronary heart disease suggests in Indo-Mauritians a susceptibility locus on chromosome 16p13 and replicates linkage with the metabolic syndrome on 3q27. Hum Mol Genet. 10:2751-65.
Franz W M, Muller O J, Katus H A. 2001. Cardiomyopathies: from genetics to the prospect of treatment. Lancet. 358:1627-37.
Fu L, Jin H, Song K, Zhang C, Shen J, Huang Y. 2001. Relationship between gene polymorphism of the PAI-1 promoter and myocardial infarction. Chin Med J (Engl). 114:266-9.
Gardemann A, Lohre J, Katz N, Tillmanns H, Hehrlein F W, Haberbosch W. 1999. The 4G4G genotype of the plasminogen activator inhibitor 4G/5G gene polymorphism is associated with coronary atherosclerosis in patients at high risk for this disease. Thromb Haemost. 82:1121-6.
Geever R F, Wilson L B, Nallaseth F S, Milner P F, Bittner M, Wilson J T. Direct identification of sickle cell anemia by blot hybridization. 1981. Proc Natl Acad Sci USA. 78:5081-5.
Georges J L, Loukaci V, Poirier O, Evans A, Luc G, Arveiler D, Ruidavets J B, Cambien F, Tiret L. 2001. Interleukin-6 gene polymorphisms and susceptibility to myocardial infarction: the ECTIM study. Etude Cas-Temoin de l'Infarctus du Myocarde. J Mol Med. 79:300-5.
Gibbs R A, Nguyen P N, Caskey C T. 1989. Detection of single DNA base differences by competitive oligonucleotide priming. Nucleic Acids Res. 17:2437-48.
Gold B. 2003. Origin and utility of the reverse dot-blot. Expert Rev Mol Diagn. 3:143-52. Gomes A V, Potter J D. Molecular and cellular aspects of troponin cardiomyopathies. Ann NY Acad Sci 2004;1015:214-24.
Griffiths A D, Malmqvist M, Marks J D, Bye J M, Embleton M J, McCafferty J, Baier M, Holliger K P, Gorick B D, Hughes-Jones N C, et al. 1993. Human anti-self antibodies with high specificity from phage display libraries. EMBO J. 12:725-34.
Guatelli J C, Whitfield K M, Kwoh D Y, Barringer K J, Richman D D, Gingeras T R. 1990. Isothermal, in vitro amplification of nucleic acids by a multienzyme reaction modeled after retroviral replication. Proc Natl Acad Sci USA. 87:1874-8.
Harrap S B, Zammit K S, Wong Z Y, Williams F M, Bahlo M, Tonkin A M, Anderson S T. 2002. Genome-wide linkage analysis of the acute coronary syndrome suggests a locus on chromosome 2. Arterioscler Thromb Vasc Biol. 22:874-8.
Hawe E, Talmud P J, Miller G J, Humphries S E; Second Northwick Park Heart Study. 2003. Family history is a coronary heart disease risk factor in the Second Northwick Park Heart Study. Ann Hum Genet. 67:97-106.
Hay B N, Sorge J A, Shopes B. 1992. Bacteriophage cloning and Escherichia coli expression of a human IgM Fab. Hum Antibodies Hybridomas. 3:81-5.
Hayashi N, Kipriyanov S, Fuchs P, Welschof M, Dorsam H, Little M. 1995. A single expression system for the display, purification and conjugation of single-chain antibodies. Gene. 160:129-30.
Helene C, Thuong N T, Harel-Bellan A. 1992. Control of gene expression by triple helix-forming oligonucleotides. The antigene strategy. Ann NY Acad Sci. 660:27-36.
Helene C. 1991. The anti-gene strategy: control of gene expression by triplex-forming-oligonucleotides. Anticancer Drug Des. 6:569-84.
Heller R F, Kelson M C. 1983. Family history in “low risk” men with coronary heart disease. J Epidemiol Community Health 37: 29-31.
Hering S, Karawajew L, Pasternak G. 1988. Raji-K562 hybrids and their use for trioma production. Biomed Biochim Acta. 47:211-6.
Hibi K, Ishigami T, Tamura K, Mizushima S, Nyui N, Fujita T, Ochiai H, Kosuge M, Watanabe Y, Yoshii Y, Kihara M, Kimura K, Ishii M, Umemura S. 1998. Endothelial nitric oxide synthase gene polymorphism and acute myocardial infarction. Hypertension. 32:521-6.
Hingorani A D, Liang C F, Fatibene J, Lyon A, Monteith S, Parsons A, Haydock S, Hopper R V, Stephens N G, O'Shaughnessy K M, Brown M J. 1999. A common variant of the endothelial nitric oxide synthase (Glu298→Asp) is a major risk factor for coronary artery disease in the UK. Circulation. 100:1515-20.
Holm J, Hillarp A, Zoller B, Erhardt L, Berntorp E, Dahlback B. 1999. Factor V Q506 (resistance to activated protein C) and prognosis after acute coronary syndrome. Thromb Haemost. 81:857-60.
Hopkins P N, Williams R R, Kuida H, Stults B M, Hunt S C, Barlow G K, Ash K O. 1988. Family history as an independent risk factor for incident coronary artery disease in a high-risk cohort in Utah. Am J Cardiol 62: 703-7.
Huber M, Mundlein A, Dornstauder E, Schneeberger C, Tempfer C B, Mueller M W, Schmidt W M. 2002. Accessing single nucleotide polymorphisms in genomic DNA by direct multiplex polymerase chain reaction amplification on oligonucleotide microarrays. Anal Biochem. 303:25-33.
Humphries S E, Luong L A, Ogg M S, Hawe E, Miller G J. 2001. The interleukin-6-174 G/C promoter polymorphism is associated with risk of coronary heart disease and systolic blood pressure in healthy men. Eur Heart J. 22:2243-52.
Humphries S E, Martin S, Cooper J, Miller G. 2002. Interaction between smoking and the stromelysin-1 (MMP3) gene 5A/6A promoter polymorphism and risk of coronary heart disease in healthy men. Ann Hum Genet. 66:343-52.
Humphries S E, Talmud P J, Hawe E, Bolla M, Day I N, Miller G J. 2001. Apolipoprotein E4 and coronary heart disease in middle-aged men who smoke: a prospective study. Lancet. 358:115-9.
Humphries S E, Talmud P J, Hawe E, Bolla M, Day I N, Miller G J. 2001. Apolipoprotein E4 and coronary heart disease in middle-aged men who smoke: a prospective study. Lancet. 358:115-9.
Huse W D, Sastry L, Iverson S A, Kang A S, Alting-Mees M, Burton D R, Benkovic S J, Lerner R A. 1989. Generation of a large combinatorial library of the immunoglobulin repertoire in phage lambda. Science. 246:1275-81.
Inbal A, Freimark D, Modan B, Chetrit A, Matetzky S, Rosenberg N, Dardik R, Baron Z, Seligsohn U. 1999. Synergistic effects of prothrombotic polymorphisms and atherogenic factors on the risk of myocardial infarction in young males. Blood. 93:2186-90.
Iwahana H, Yoshimoto K, Itakara M. 1992. Detection of point mutations by SSCP of PCR-amplified DNA after endonuclease digestion. Biotechniques. 12:64-66.
Jaeger L B, Banks W A. 2004. Antisense therapeutics and the treatment of CNS disease. Front Biosci. 9:1720-7.
Jiricny J, Su S S, Wood S G, Modrich P. 1988. Mismatch-containing oligonucleotide duplexes bound by the E. coli mutS-encoded protein. Nucleic Acids Res. 16:7843-53.
Jousilahti P, Puska P, Vartiainen E, Pekkanen J, Tuomilehto J. 1996. Parental history of premature coronary heart disease: an independent risk factor of myocardial infarction. J Clin Epidemiol 49: 497-503.
Kim R J, Becker R C. 2003. Association between factor V Leiden, prothrombin G20210A, and methylenetetrahydrofolate reductase C677T mutations and events of the arterial circulatory system: a meta-analysis of published studies. Am Heart J. 146:948-57.
Kohler G, Milstein C. 1975. Continuous cultures of fused cells secreting antibody of predefined specificity. Nature 256:495-497.
Kozbor D, Lagarde A E, Roder J C. 1982. Human hybridomas constructed with antigen-specific Epstein-Barr virus-transformed cell lines. Proc Natl Acad Sci USA. 79:6651-5.
Kumar P, Luthra K, Dwivedi M, Behl V K, Pandey R M, Misra A. 2003. Apolipoprotein E gene polymorphisms in patients with premature myocardial infarction: a case-controlled study in Asian Indians in North India. Ann Clin Biochem. 40:382-7.
Kwoh D Y, Davis G R, Whitfield K M, Chappelle H L, DiMichele L J, Gingeras T R. 1989. Transcription-based amplification system and detection of amplified human immunodeficiency virus type 1 with a bead-based sandwich hybridization format. Proc Natl Acad Sci USA. 86:1173-7.
Kwok P-Y. 2001. Methods for genotyping single nucleotide polymorphisms. Ann Rev Genomics Hum Genet. 2:235-258.
Landegren U, Kaiser R, Sanders J, Hood L. 1988. A ligase-mediated gene detection technique. Science. 241:1077-80.
Lemaitre M, Bayard B, Lebleu B. 1987. Specific antiviral activity of a poly(L-lysine)-conjugated oligodeoxyribonucleotide sequence complementary to vesicular stomatitis virus N protein mRNA initiation site. Proc Natl Acad Sci USA. 84:648-52.
Letsinger R L, Zhang G R, Sun D K, Ikeuchi T, Sarin P S. 1989. Cholesteryl-conjugated oligonucleotides: synthesis, properties, and activity as inhibitors of replication of human immunodeficiency virus in cell culture. Proc Natl Acad Sci USA. 86:6553-6.
Li R, Bensen J T, Hutchinson R G, Province M A, Hertz-Picciotto I, Sprafka J M, Tyroler H A. 2000. Family risk score of coronary heart disease (CHD) as a predictor of CHD: the Atherosclerosis Risk in Communities (ARIC) study and the NHLBI family heart study. Genet Epidemiol 18: 236-50.
Li Y H, Chen J H, Tsai W C, Chao T H, Guo H R, Tsai L M, Wu H L, Shi G Y. 2002. Synergistic effect of thrombomodulin promoter -33G/A polymorphism and smoking on the onset of acute myocardial infarction. Thromb Haemost. 87:86-91.
Libby P. 1995. Molecular bases of the acute coronary syndromes. Circulation 91: 2844-50.
Marenberg M E, Risch N, Berkman L F, Floderus B, de Faire U. 1994. Genetic susceptibility to death from coronary heart disease in a study of twins. N Engl J Med. 330:1041-6.
Meade H, Gates L, Lacy E, Lonberg N. 1990. Bovine alpha S1-casein gene sequences direct high level expression of active human urokinase in mouse milk. Biotechnology (NY). 8:443-6.

Mikkelsson J, Perola M, Wartiovaara U, Peltonen L, Palotie A, Penttila A, Karhunen P J. 2000. Plasminogen activator inhibitor-1 (PAI-1) 4G/5G polymorphism, coronary thrombosis, and myocardial infarction in middle-aged Finnish men who died suddenly. Thromb Haemost. 84:78-82.
Murray C J L, Lopez A D. 1997. Global mortality, disability, and the contribution of risk factors: Global Burden of Disease Study. Lancet 349:1436-42.
Mustafina O E, Shagisultanova E I, Tuktarova I A, Khusnutdinova E K. 2002. Polymorphism of the apolipoprotein E gene and the risk of myocardial infarction. Mol Biol (Mosk). 36:978-84.
Myers R H, Kiely D K, Cupples L A, Kannel W B. 1990. Parental history is an independent risk factor for coronary artery disease: the Framingham Study. Am Heart J 120: 963-9.
Myers R M, Fischer S G, Lerman L S, Maniatis T. 1985. Nearly all single base substitutions in DNA fragments joined to a GC-clamp can be detected by denaturing gradient gel electrophoresis. Nucleic Acids Res. 13:3131-45.
Myers R M, Larin Z, Maniatis T. 1985. Detection of single base substitutions by ribonuclease cleavage at mismatches in RNA: DNA duplexes. Science. 230:1242-6.
Nakai K, Fusazaki T, Zhang T, Shiroto T, Osawa M, Kamata J, Itoh M, Nakai K, Habano W, Kiuchi T, Yamamori S, Hiramori K. 1998. Polymorphism of the apolipoprotein E and angiotensin I converting enzyme genes in Japanese patients with myocardial infarction. Coron Artery Dis. 9:329-34.
Nielsen P E, Egholm M, Berg R H, Buchardt O. 1991. Sequence-selective recognition of DNA by strand displacement with a thymine-substituted polyamide. Science. 254:1497-500.
Norlund L, Holm J, Zoller B, Ohlin A K. 1997. A common thrombomodulin amino acid dimorphism is associated with myocardial infarction. Thromb Haemost. 77:248-51.
Orita M, Iwahana H, Kanazawa H, Hayashi K, Sekiya T. 1989. Detection of polymorphisms of human DNA by gel electrophoresis as single-strand conformation polymorphisms. Proc Natl Acad Sci USA. 86:2766-70.
Pajukanta P, Cargill M, Viitanen L, Nuotio I, Kareinen A, Perola M, Terwilliger J D, Kempas E, Daly M, Lilja H, Rioux J D, Brettin T, Viikari J S, Ronnemaa T, Laakso M, Lander E S, Peltonen L. 2000. Two loci on chromosomes 2 and X for premature coronary heart disease identified in early- and late-settlement populations of Finland. Am J Hum Genet. 67:1481-93.
Park H Y, Nabika T, Jang Y, Kwon H M, Cho S Y, Masuda J. 2002. Association of G-33A polymorphism in the thrombomodulin gene with myocardial infarction in Koreans. Hypertens Res. 25:389-94.
Park J E, Lee W H, Hwang T H, Chu J A, Kim S, Choi Y H, Kim J S, Kim D K, Lee S H, Hong K P, Seo J D, Lee W R. 2000. Aging affects the association between endothelial nitric oxide synthase gene polymorphism and acute myocardial infarction in the Korean male population. Korean J Intern Med. 15:65-70.
Pastinen T, Perola M, Niini P, Terwilliger J, Salomaa V, Vartiainen E, Peltonen L, Syvanen A. 1998. Array-based multiplex analysis of candidate genes reveals two independent and additive genetic risk factors for myocardial infarction in the Finnish population. Hum Mol Genet. 7:1453-62.
Peltonen L, Jalanko A, Varilo T. Molecular genetics of the Finnish disease heritage. 1999. Hum Mol Genet. 8:1913-23.
Petersen M, Wengel J. 2003. LNA: a versatile tool for therapeutics and genomics. Trends Biotechnol. 21:74-81.
Rauramaa R, Vaisanen S B, Luong L A, Schmidt-Trucksass A, Penttila I M, Bouchard C, Toyry J, Humphries S E. 2000. Stromelysin-1 and interleukin-6 gene promoter polymorphisms are determinants of asymptomatic carotid artery atherosclerosis. Arterioscler Thromb Vasc Biol. 20:2657-62.
Rewers M, Kamboh M I, Hoag S, Shetterly S M, Ferrell R E, Hamman R F. 1994. ApoA-IV polymorphism associated with myocardial infarction in obese NIDDM patients. The San Luis Valley Diabetes Study. Diabetes. 43:1485-9.
Ross R. 1999. Atherosclerosis—an inflammatory disease. Engl J Med. 340:115-26.
Rundek T, Elkind M S, Pittman J, Boden-Albala B, Martin S, Humphries S E, Juo S H, Sacco R L. 2002. Carotid intima-media thickness is associated with allelic variants of stromelysin-1, interleukin-6, and hepatic lipase genes: the Northern Manhattan Prospective Cohort Study. Stroke. 33:1420-3.
Saiki R K, Bugawan T L, Horn G T, Mullis K B, Erlich H A. 1986. Analysis of enzymatically amplified beta-globin and HLA-DQ alpha DNA with allele-specific oligonucleotide probes. Nature. 324:163-6.
Sajantila A, Salem A H, Savolainen P, Bauer K, Gierig C, Paabo S. 1996. Paternal and maternal DNA lineages reveal a bottleneck in the founding of the Finnish population. Proc Natl Acad Sci USA. 93: 12035-9.
Salonen J T, Malin R, Tuomainen T P, Nyyssonen K, Lakka T A, Lehtimaki T. 1999. Polymorphism in high density lipoprotein paraoxonase gene and risk of acute myocardial infarction in men: prospective nested case-control study. BMJ. 319:487-9.
Salonen J T, Salonen R, Seppänen K, Rauramaa R, Tuomilehto J. 1991. High density lipoprotein, HDL2 and HDL3 subfractions and the risk of acute myocardial infarction: a prospective population study in Eastern Finnish men. Circulation 84:129-139.
Salonen J T, Ylä-Herttuala S, Yamamoto R, Butler S, Korpela H, Salonen R, Nyyssönen K, Palinski W, Witztum J L. 1992. Autoantibody against oxidised LDL and progression of carotid atherosclerosis. Lancet 339:883-887.
Salonen J T. 1988. Is there a continuing need for longitudinal epidemiologic research? The Kuopio Ischaemic Heart Disease Risk Factor Study. Ann Clin Res 20: 46-50.
Sanghera D K, Saha N, Aston C E, Kamboh M I. 1997. Genetic polymorphism of paraoxonase and the risk of coronary heart disease. Arterioscler Thromb Vasc Biol. 17:1067-73.
Sen-Baneree S, Siles X, Campos H. 2000. Tobacco smoking modifies association between Gln-Arg192 polymorphism of human paraoxonase gene and risk of myocardial infarction. Arterioscler Thromb Vasc Biol. 20:2120-6.
Senti M, Tomas M, Vila J, Marrugat J, Elosua R, Sala J, Masia R. 2001. Relationship of age-related myocardial infarction risk and Gln/Arg 192 variants of the human paraoxonase1 gene: the REGICOR study. Atherosclerosis. 156:443-9.
Serrato M, Marian A J. 1995. A variant of human paraoxonase/arylesterase (HUMPONA) gene is a risk factor for coronary artery disease. J Clin Invest. 96:3005-8.
Sevon P. 2004. Algorithms for Association-Based Gene Mapping. PhD Thesis, Series of Publication A, Report A-2004-4, University of Helsinki, Department of Computer Science, 101 pages, ISBN 952-10-1926-3 (pdf).
Sheffield V C, Cox D R, Lerman L S, Myers R M. 1989. Attachment of a 40-base-pair G+C−rich sequence (GC-clamp) to genomic DNA fragments by the polymerase chain reaction results in improved detection of single-base changes. Proc Natl Acad Sci USA. 86:232-6.
Shimasaki Y, Yasue H, Yoshimura M, Nakayama M, Kugiyama K, Ogawa H, Harada E, Masuda T, Koyama W, Saito Y, Miyamoto Y, Ogawa Y, Nakao K. 1998. Association of the missense Glu298Asp variant of the endothelial nitric oxide synthase gene with myocardial infarction. J Am Coll Cardiol. 31:1506-10.
Sholtz R I, Rosenman R H, Brand R J. 1975. The relationship of reported parental history to the incidence of coronary heart disease in the Western Collaborative Group Study. Am J Epidemiol 102: 350-6.
Simon R. 2003. Using DNA microarrays for diagnostic and prognostic prediction. Expert Rev Mol Diagn. 3:587-95.
Smith D B, Flavell R B. 1977. Nucleotide sequence organisation in the rye genome. Biochim Biophys Acta. 474:82-97.
Smithies O, Gregg R G, Boggs S S, Koralewski M A, Kucherlapati R S. 1985. Insertion of DNA sequences into the human chromosomal beta-globin locus by homologous recombination. Nature. 317:230-4.
Stamler J, Greenland P, Neaton J D. 1998. The established major risk factors underlying epidemic coronary and cardiovascular disease. CVD Prevention 1: 82-97.
Stary H C, Blankenhorn D H, Chandler A B, Glagov S, Insull W Jr, Richardson M, Rosenfeld M E, Schaffer S A, Schwartz C J, Wagner W E, et al. 1992. A definition of the intima of human arteries and of its atherosclerosis-prone regions. A report from the Committee on Vascular Lesions of the Council on Arteriosclerosis, American Heart Association. Circulation 85: 391-405.
Stary H C, Chandler A B, Dinsmore R E, Fuster V, Glagov S, Insull W Jr, Rosenfeld M E, Schwartz C J, Wagner W D, Wissler R W. 1995. A definition of advanced types of atherosclerotic lesions and a histological classification of atherosclerosis. A report from the Committee on Vascular Lesions of the Council on Arteriosclerosis, American Heart Association. Circulation 92: 1355-74.
Stary H C, Chandler A B, Glagov S, Guyton J R, Insull W Jr, Rosenfeld M E, Schaffer S A, Schwartz C J, Wagner W D, Wissler R W. 1994. A definition of initial, fatty streak, and intermediate lesions of atherosclerosis. A report from the Committee on Vascular Lesions of the Council on Arteriosclerosis, American Heart Association. Circulation 89: 2462-78.
Stary H C. 2000. Lipid and macrophage accumulations in arteries of children and the development of atherosclerosis. Am J Clin Nutr 72(5 Suppl): S1297-1306.
Stein C A, Cohen J S. 1988. Oligodeoxynucleotides as inhibitors of gene expression: a review. Cancer Res. 48:2659-68.
Steinberg D. Lewis A. 1997. Conner Memorial Lecture. Oxidative modification of LDL and atherogenesis. Circulation 95: 1062-71.
Stephens J W, Humphries S E. 2003. The molecular genetics of cardiovascular disease: clinical implications. J Intern Med. 253:120-7.
Syvänen A-C. 2001. Accessing genetic variation: Genotyping single nucleotide polymorphisms. Nature Reviews Genetics. 2:930-942.
Terashima M, Akita H, Kanazawa K, Inoue N, Yamada S, Ito K, Matsuda Y, Takai E, Iwai C, Kurogane H, Yoshida Y, Yokoyama M. 1999. Stromelysin promoter 5A/6A polymorphism is associated with acute myocardial infarction. Circulation. 99:2717-9.
Thomas K R, Capecchi M R. 1987. Site-directed mutagenesis by gene targeting in mouse embryo-derived stem cells. Cell. 51:503-12.
Thompson S, Clarke A R, Pow A M, Hooper M L, Melton D W. 1989. Germ line transmission and expression of a corrected HPRT gene produced by gene targeting in embryonic stem cells. Cell. 56:313-21.
Toivonen H T, Onkamo P, Vasko K, Ollikainen V, Sevon P, Mannila H, Herr M, Kere J. 2000. Data mining applied to linkage disequilibrium mapping. Am J Hum Genet. 67:133-45.
Tremoli E, Camera M, Toschi V, Colli S. 1999. Tissue factor in atherosclerosis. Atherosclerosis. 144:273-83.
Tun A, Khan I A. Myocardial infarction with normal coronary arteries: the pathologic and clinical perspectives. Angiology. 2001; 52:299-304.
Tuomainen T P, Kontula K, Nyyssonen K, Lakka T A, Helio T, Salonen J T. 1999. Increased risk of acute myocardial infarction in carriers of the hemochromatosis gene Cys282Tyr mutation: a prospective cohort study in men in eastern Finland. Circulation. 100: 1274-9.
Waller B F, Fry E T, Hermiller J B, Peters T, Slack J D. 1996. Nonatherosclerotic causes of coronary artery narrowing—Part II. Clin Cardiol.19:587-91.
Waller B F, Fry E T, Hermiller J B, Peters T, Slack J D. 1996. Nonatherosclerotic causes of coronary artery narrowing—Part I. Clin Cardiol. 19:509-12.
Waller B F, Fry E T, Hermiller J B, Peters T, Slack J D. 1996. Nonatherosclerotic causes of coronary artery narrowing—Part III. Clin Cardiol. 19:656-61.
van der Krol A R, Mol J N, Stuitje A R. 1988. Modulation of eukaryotic gene expression by complementary RNA or DNA-sequences. Biotechniques 6:958-76.
Wang Q, Rao S, Shen G Q, Li L, Moliterno D J, Newby L K, Rogers W J, Cannata R, Zirzow E, Elston R C, Topol E J. 2004. Premature myocardial infarction novel susceptibility locus on chromosome 1P34-36 identified by genomewide linkage analysis. Am J Hum Genet. 74:262-71.
Wei D, Shan J, Chen Z, Shi Y. 2002. The G894T mutation of the endothelial nitric oxide synthase gene is associated with coronary atherosclerotic heart disease in Chinese. Zhonghua Yi Xue Yi Chuan Xue Za Zhi. 19:471-4.
Virmani R, Burke A P, Farb A. 2001. Sudden cardiac death. Cardiovasc Pathol.10:211-8.
Wong W M, Hawe E, Li L K, Miller G J, Nicaud V, Pennacchio L A, Humphries S E, Talmud P J. 2003. Apolipoprotein AIV gene variant S347 is associated with increased risk of coronary heart disease and lower plasma apolipoprotein AIV levels. Circ Res. 92:969-75.
Wood D. 2001. Established and emerging cardiovascular risk factors. Am Heart J 141(2 Suppl): S49-57.
World Health Organization.2004. Cardiovascular diseases: prevention and control. Information sheet. http://www.who.int/dietphysicalactivity/media/en/gsfs_cvd.pdf (Accessed 14.07.04)
Wu K K, Aleksic N, Ahn C, Boerwinkle E, Folsom A R, Juneja H; Atherosclerosis Risk in Communities Study (ARIC) Investigators. 2001. Thrombomodulin Ala455Val polymorphism and risk of coronary heart disease. Circulation. 103:1386-9.
Zhan M, Zhou Y, Han Z. 2003. Plasminogen activator inhibitor-1 4G/5G gene polymorphism in patients with myocardial or cerebrovascular infarction in Tianjin, China. Chin Med J. 116:1707-10.
Zon G. 1988. Oligonucleotide analogues as potential chemotherapeutic agents. Pharm Res. 5:539-49.
Zon G. 1988. Oligonucleotide analogues as potential chemotherapeutic agents. Pharm Res. 5:539-49.

1. A method for identification of an individual who has an altered risk of or susceptibility for developing AMI, the method comprising the steps of:
a) providing a biological sample taken from said individual;
b) collecting personal and clinical information of said individual;
c) determining the nucleotides present in one or several of the polymorphic sites as set forth in tables 3, 4, 5, 6, 7, 8, 10 and 11 in said individual's nucleic acid; and
d) combining the SNP marker data with personal and clinical information to assess the risk of an individual to develop AMI.

2. The method according to claim 1, wherein the altered risk is an increased risk of AMI.

3. The method according to claim 1, wherein the altered risk is a decreased risk of AMI.

4. The method according to claim 1, wherein the polymorphic sites are those present in the haplotypes presented in tables 4, 5, 6, 7, 8, 10 and 11.

5. The method according to claim 1, wherein the polymorphic sites are associated with the SNP markers set forth in tables 3, 4, 5, 6, 7, 8, 10 and 11.

6. The method according to claim 5, wherein the polymorphic sites are in complete linkage disequilibrium with the SNP markers set forth in tables 3, 4, 5, 6, 7, 8, 10 and 11.

7. The method according to claim 6, wherein the polymorphic sites are in complete linkage disequilibrium in the population in which the said method is used.

8. A method for identification of an individual who has an altered risk of or susceptibility for developing AMI, the method comprising the steps of
a) providing a biological sample taken from a subject
b) determining the nucleotides present in one or several of the polymorphic sites as set forth in tables 3, 4, 5, 6, 7, 8, 10 and 11 in said individual's nucleic acid
c) combining the SNP marker data to assess the risk of an individual to develop AMI

9. The method according to claim 8, wherein the altered risk is an increased risk of AMI.

10. The method according to claim 8, wherein the altered risk is a decreased risk of AMI.

11. The method according to claim 8, wherein the polymorphic sites are those present in the haplotypes presented in tables 4, 5, 6, 7, 8, 10 and 11.

12. The method according to claim 8, wherein the polymorphic sites are associated with the SNP markers set forth in tables 3, 4, 5, 6, 7, 8, 10 and 11.

13. The method according to claim 12, wherein the polymorphic sites are in complete linkage disequilibrium with the SNP markers set forth in tables 3, 4, 5, 6, 7, 8, 10 and 11.

14. The method according to claim 13, wherein the polymorphic sites are in complete linkage disequilibrium in the population in which the said method is used.

15. The method according to claim 1, wherein said one or several polymorphic sites reside within an AMI risk gene or genes as set forth in table 9.

16. The method according to claim 1, wherein the AMI risk genes reside in the genome region which is defined by the haplotype pattern mining analysis, the genes set forth in tables 4, 5, 6, 10 and 11

17. The method according to claim 1, wherein the polymorphic sites are associated with the haplotype regions, haplotypes or SNP markers defining the haplotypes set forth in tables 4, 5, 6, 7, 8, 10and 11.

18. The method according to claim 17, wherein the polymorphic sites are in complete linkage disequilibrium with the haplotype regions, haplotypes or SNP markers defining the haplotypes set forth in tables 4, 5, 6, 7, 8, 10 and 11.

19. The method according to claim 18, wherein the polymorphic sites are in complete linkage disequilibrium in the population in which the said method is used.

20. The method according to claim 1, wherein one or several of the SNP markers are selected from the group consisting of the following haplotypes and individual SNPs:
a) rs10515495 (A/G) (SEQ ID NO:509), rs4976445 (A/G) (SEQ ID NO:1451), rs10491335 (A/G) (SEQ ID NO:148), and rs6878439 (A/G) (SEQ ID NO:1584) defining the protective haplotype GAGG (or nucleotides from the complementary strand);
b) rs10486559 (A/G) (SEQ ID NO:88), rs10486562 (A/G) (SEQ ID NO:89), rs757397 (C/T) (SEQ ID NO:1717), rs735664 (C/T) (SEQ ID NO:1695), and rs720659 (G/T) (SEQ ID NO:1656) defining the risk haplotype AATTT (or nucleotides from the complementary strand);
c) rs10503555 (A/G) (SEQ ID NO:347), rs10503557 (C/T) (SEQ ID NO:348), rs2410361 (A/C) (SEQ ID NO:1159), and rs10503561 (C/T) (SEQ ID NO:350) defining the protective haplotype GCCC (or nucleotides from the complementary strand);
d) rs260816 (C/G) (SEQ ID NO:1188), rs260818 (A/C) (SEQ ID NO:1189), and rs361315 (A/G) (SEQ ID NO:1300) defining the protective haplotype GCA (or nucleotides from the complementary strand);
e) rs762721 (A/C) (SEQ ID NO:1729), rs10491992 (C/G) (SEQ ID NO:170), rs10491993 (A/G) (SEQ ID NO:171), and rs2213177 (A/G) (SEQ ID NO:1084) defining the protective haplotype CGAA or nucleotides from the complementary strand);
f) rs10509423 (C/T) (SEQ ID NO:402) defining the risk allele homozygote to T;
g) rs1004361 (A/G) (SEQ ID NO:3) defining the risk allele carrier A;
h) rs10483262 (A/C) (SEQ ID NO:51) defining the risk allele carrier A;
i) rs7342999 (A/G) (SEQ ID NO:1694) defining the risk allele carrier G;
j) rs1554472 (C/T) (SEQ ID NO:851) defining the risk allele carrier T;
k) rs9294536 (A/C) (SEQ ID NO:1905) defining the protective allele carrier A and
l) rs1125465 (A/G) (SEQ ID NO:627) defining the protective allele carrier A

21-32. (canceled)

33. The method according to claim 1, wherein one or several of the SNP markers are selected from the group consisting of the following haplotypes or individual SNPs:
a) rs834485 (C/T) (SEQ ID NO:1830), rs856283 (A/G) (SEQ ID NO:1846), rs1260817 (A/C) (SEQ ID NO:653), rs1260772 (A/G) (SEQ ID NO:652) defining the haplotype T G C A (or nucleotides from the complementary strand);
b) rs7605386 (C/G) (SEQ ID NO:1727), and rs9288697 (A/C) (SEQ ID NO:1891) defining the haplotype G A (or nucleotides from the complementary strand);
c) rs10483879 (C/T) (SEQ ID NO:57), rs2885625 (A/G) (SEQ ID NO:1254), rs6574333 (A/G) (SEQ ID NO:1551) defining the haplotype C G A (or nucleotides from the complementary strand);
d) rs223128 (G/T) (SEQ ID NO:1098), rs223154 (G/T) (SEQ ID NO:1099), rs315499 (A/G) (SEQ ID NO:1281), rs10512437 (A/G) (SEQ ID NO:453) defining the haplotype G T G G (or nucleotides from the complementary strand);
e) rs799275 (C/T) (SEQ ID NO:1805), rs5957801 (A/G) (SEQ ID NO:1492), rs5909851 (A/G) (SEQ ID NO:1486), rs10521707 (A/G) SEQ ID NO:603 defining the haplotype T G G A (or nucleotides from the complementary strand);
f) rs953304 (C/G) (SEQ ID NO:1991) defining the risk allele carrier G
g) rs1881922 (C/T) (SEQ ID NO:926) defining the risk allele carrier C
h) rs10486619 (C/T) (SEQ ID NO:91) defining the risk allele carrier C
i) rs192714 (C/T) (SEQ ID NO:950) defining the risk allele homozygote to C
j) rs7100162 (C/T) (SEQ ID NO:1619) defining the risk allele carrier C
k) rs9301916 (C/G) (SEQ ID NO:1913) defining the risk allele homozygote to C
l) rs6564876 (C/G) (SEQ ID NO:1547) defining the risk allele homozygote to C

34-46. (canceled)

47. The method according to claim 1, wherein one or several of the SNP markers are selected from the group consisting of the following haplotypes:
a) rs834485 (C/T) (SEQ ID NO:1830), rs856283 (A/G) (SEQ ID NO:1846), rs1260817 (A/C) (SEQ ID NO:653), and rs1260772 (A/G) (SEQ ID NO:652) defining the haplotype T G C A (or nucleotides from the complementary strand);
b) rs1932818 (C/T) (SEQ ID NO:952), rs6663269 (C/G) (SEQ ID NO:1560) defining the haplotype C C (or nucleotides from the complementary strand);
c) rs7605386 (C/G) (SEQ ID NO:1727), and rs9288697 (A/C) (SEQ ID NO:1891) defining the haplotype G A (or nucleotides from the complementary strand);
d) rs3903306 (A/T) (SEQ ID NO:1342), rs6432539 (C/T) (SEQ ID NO:1526), rs1364703 (C/G) (SEQ ID NO:706), rs1966530 (A/G) (SEQ ID NO:976), rs5002908 (A/G) (SEQ ID NO:1455) defining the haplotype A T G G A (or nucleotides from the complementary strand);
e) rs997274 (C/T) (SEQ ID NO:2035), rs6788511 (A/C) (SEQ ID NO:1574) defining the haplotype T C (or nucleotides from the complementary strand);
f) rs864391 (C/T) (SEQ ID NO:1856), rs854202 (A/G) (SEQ ID NO:1842), rs2014378 (A/G) (SEQ ID NO:996), rs952621 (A/G) (SEQ ID NO:1985), rs1877960 (C/T) (SEQ ID NO:922) defining the haplotype T A A G C (or nucleotides from the complementary strand);
g) rs1445362 (A/G) (SEQ ID NO:793), rs10513252 (A/G) (SEQ ID NO:470), rs10513253 (C/T) (SEQ ID NO:471), rs4610179 (A/G) (SEQ ID NO:1395) defining the haplotype A A T G (or nucleotides from the complementary strand);
h) rs682913 (C/T) (SEQ ID NO:1581), rs725425 (A/C) (SEQ ID NO:1674), rs1423260 (A/G) (SEQ ID NO:768) defining the haplotype T A G (or nucleotides from the complementary strand);
i) rs919740 (C/T) (SEQ ID NO:1868), rs10515639 (A/G) (SEQ ID NO:511) defining the haplotype C A (or nucleotides from the complementary strand);
j) rs10499001 (C/T) (SEQ ID NO:265), rs4144270 (A/G) (SEQ ID NO:1368), rs4515397 (A/G) (SEQ ID NO:1385), rs10499000 (C/T) (SEQ ID NO:264) defining the haplotype T A G T (or nucleotides from the complementary strand);
k) rs1407658 (C/T) (SEQ ID NO:749), rs2025272 (G/T) (SEQ ID NO:1005), rs9283864 (C/G) (SEQ ID NO:1880) defining the haplotype C T G (or nucleotides from the complementary strand);
l) rs10503268 (A/C) (SEQ ID NO:332), rs1038062 (C/T) (SEQ ID NO:41), rs10503269 (A/G) (SEQ ID NO:333), rs1038058 (C/T) (SEQ ID NO:40) defining the haplotype A C G T (or nucleotides from the complementary strand);
m) rs10503616 (C/T) (SEQ ID NO:352), rs10503617 (C/T) (SEQ ID NO:353), rs2717719 (C/T) (SEQ ID NO:1213), rs1488925 (A/G) (SEQ ID NO:816), rs2035681 (C/T) (SEQ ID NO:1009) defining the haplotype C C C G C (or nucleotides from the complementary strand);
n) rs2994298 (C/T) (SEQ ID NO:1271), rs7463074 (A/G) (SEQ ID NO:1706), rs2975534 (C/T) (SEQ ID NO:1267), rs2975533 (C/T) (SEQ ID NO:1266) defining the haplotype C G T C (or nucleotides from the complementary strand);
o) rs10491744 (A/G) (SEQ ID NO:159), rs1543587 (A/G) (SEQ ID NO:844), rs1074789 (C/T) (SEQ ID NO:623), rs7025842 (C/T) (SEQ ID NO:1605) defining the haplotype G G T T (or nucleotides from the complementary strand);
p) rs10491753 (A/C) (SEQ ID NO:161), rs13284133 (A/C) (SEQ ID NO:676), rs10491756 (G/T) (SEQ ID NO:162), rs10491757 (A/G) (SEQ ID NO:163) defining the haplotype A C T A (or nucleotides from the complementary strand);
q) rs1542750 (A/G) (SEQ ID NO:843), rs10501763 (C/T) (SEQ ID NO:299), rs10501764 (A/G) (SEQ ID NO:300), rs10501765 (C/T) (SEQ ID NO:301) defining the haplotypeG T G C (or nucleotides from the complementary strand);
r) rs721346(A/C) (SEQ ID NO:1664), rs1400549(C/T) (SEQ ID NO:739), rs503208 (C/G) (SEQ ID NO:1458), rs7127296 (A/C) (SEQ ID NO:1633) defining the haplotype A T C C (or nucleotides from the complementary strand);
s) rs10483879 (C/T) (SEQ ID NO:57), rs2885625 (A/G) (SEQ ID NO:1254), rs6574333 (A/G) (SEQ ID NO:1551) defining the haplotype C G A (or nucleotides from the complementary strand);
t) rs1049884 (A/T) (SEQ ID NO:256), rs1364256 (C/T) (SEQ ID NO:705), rs7185078 (C/T) (SEQ ID NO:1648), rs7193075 (A/G) (SEQ ID NO:1652) defining the haplotype A T T A (or nucleotides from the complementary strand);
u) rs10521277 (A/G) (SEQ ID NO:599), rs1019156 (A/G) (SEQ ID NO:1) defining the haplotype G G (or nucleotides from the complementary strand);
v) rs223128 (G/T ) (SEQ ID NO:1098), rs223154 (G/T) (SEQ ID NO:1099), rs315499 (A/G) (SEQ ID NO:1281), rs10512437 (A/G) (SEQ ID NO:453) defining the haplotype G T G G (or nucleotides from the complementary strand);
w) rs10513997 (C/T) (SEQ ID NO:482), rs10513998 (A/G) (SEQ ID NO:483), rs10514026 (T/G) (SEQ ID NO:486)defining the haplotype T A T (or nucleotides from the complementary strand);
x) rs799275 (C/T) (SEQ ID NO:1805), rs5957801 (A/G) (SEQ ID NO:1492), rs5909851 (A/G) (SEQ ID NO:1486), rs10521707 (A/G) (SEQ ID NO:603) defining the haplotypeT G G A (or nucleotides from the complementary strand); and
y) rs764198 (A/G) (SEQ ID NO:1735), rs10521816 (A/G) (SEQ ID NO:609), rs10521817 (C/T) (SEQ ID NO:610), rs3l135496 (C/T) (SEQ ID NO:1279) defining the haplotype G A C T (or nucleotides from the complementary strand).

48-72. (canceled)

73. The method according to claim 1, wherein one or several of the SNP markers are selected from the group consisting of the following haplotypes:
a) rs623360 (C/G) (SEQ ID NO:1513) and rs627069 (C/G) (SEQ ID NO:1515) defining the haplotype C C (or nucleotides from the complementary strand);
b) rs7547716 (A/C) (SEQ ID NO:1713), rs1891174 (C/T) (SEQ ID NO:932), and rs10494841 (A/G) (SEQ ID NO:201) defining the haplotype C T G (or nucleotides from the complementary strand);
c) rs10493353 (C/T) (SEQ ID NO:187), rs1323851 (G/T) (SEQ ID NO:672), rs834485 (C/T) (SEQ ID NO:1830), rs856283 (A/G) (SEQ ID NO:1846), and rs 1260817 (A/C) (SEQ ID NO:653) defining the haplotype C G T G C (or nucleotides from the complementary strand);
d) rs3106653 (A/C) (SEQ ID NO:1274), rs2961958 (A/T) (SEQ ID NO:1264), rs2591169 (A/T) (SEQ ID NO:1184), and rs 10497144 (C/G) (SEQ ID NO:227) defining the haplotype A T A C (or nucleotides from the complementary strand);
e) rs10497192 (C/T) (SEQ ID NO:235), rs7605386 (C/G) (SEQ ID NO:1727), and rs9288697 (A/C) (SEQ ID NO:1891) defining the haplotype C G A (or nucleotides from the complementary strand);
f) rs1851328 (A/G) (SEQ ID NO:912), rs6711457 (C/T) (SEQ ID NO:1566), rs10498053 (C/G) (SEQ ID NO:252), and rs6744504 (C/T) (SEQ ID NO:1571) defining the haplotype G C G T (or nucleotides from the complementary strand);
g) rs9310496 (A/G) (SEQ ID NO:1930), rs10510450 (A/G) (SEQ ID NO:423), rs4685356 (A/C) (SEQ ID NO:1405), and rs10510452 (A/G) (SEQ ID NO:425) defining the haplotype A G C A (or nucleotides from the complementary strand);
h) rs10510660 (A/G) (SEQ ID NO:427), rs951973 (C/T) (SEQ ID NO:1982), rs1487994 (G/T) (SEQ ID NO:815), rs10510661 (C/G) (SEQ ID NO:428), and rs6805290 (C/T) (SEQ ID NO:1576) defining the haplotype A C T G C (or nucleotides from the complementary strand);
i) rs1018341 (G/T) (SEQ ID NO:10), rs953304 (C/G) (SEQ ID NO:1991), rs10514726 (G/T) (SEQ ID NO:496) defining the haplotype G C G (or nucleotides from the complementary strand);
j) rs1355533 (C/T) (SEQ ID NO:698), rs1357287 (A/G) (SEQ ID NO:703), rs1403101 (C/T) (SEQ ID NO:740), and rs1356612 (C/T) (SEQ ID NO:700) defining the haplotype C G T C (or nucleotides from the complementary strand);
k) rs10513261 (C/T) (SEQ ID NO:472) and rs1881922 (C/T) (SEQ ID NO:926) defining the haplotype T C (or nucleotides from the complementary strand);
l) rs1023714 (A/T) (SEQ ID NO:17), rs2400502 (A/G) (SEQ ID NO:1157), rs2400503 (C/T) 1158), rs10515605 (A/G) (SEQ ID NO:510), and rs7709159 (C/T) (SEQ ID NO:1747) defining the haplotype A A C A C (or nucleotides from the complementary strand);
m) rs10515495 (A/G) (SEQ ID NO:509), rs4976445 (A/G) (SEQ ID NO:1451), rs10491335 (A/G) (SEQ ID NO:148), and rs6878439 (A/G) (SEQ ID NO:1584) defining the haplotype G A G G (or nucleotides from the complementary strand);
n) rs786135 (A/C) (SEQ ID NO:1782), rs1357194 (C/T) (SEQ ID NO:702), rs9285412 (C/T) (SEQ ID NO:1884), and rs9320498 (A/C) (SEQ ID NO:1951) defining the haplotype A C C C (or nucleotides from the complementary strand);
o) rs10486559 (A/G) (SEQ ID NO:88), rs10486562 (A/G) (SEQ ID NO:89), rs757397 (C/T) (SEQ ID NO:1717), rs735664 (C/T) (SEQ ID NO:1695), and rs720659 (G/T) (SEQ ID NO:1656) defining the haplotype A A T T T (or nucleotides from the complementary strand);
p) rs1961352 (A/C) (SEQ ID NO:974), rs2029832 (C/T) (SEQ ID NO:1007), rs9741 (C/T) (SEQ ID NO:2021), and rs10503616 (C/T) (SEQ ID NO:352) defining the haplotype C C T T (or nucleotides from the complementary strand);
q) rs10503555 (A/G) (SEQ ID NO:347), rs10503557 (C/T) (SEQ ID NO:348), rs2410361 (A/C) (SEQ ID NO:1159), and rs10503561 (C/T) (SEQ ID NO:350) defining the haplotype G C C C (or nucleotides from the complementary strand);
r) rs260816 (C/G) (SEQ ID NO:1188), rs260818 (A/C) (SEQ ID NO:1189), rs361315 (A/G) (SEQ ID NO:1300) defining the haplotype G C A (or nucleotides from the complementary strand);
s) rs1940357 (A/C) (SEQ ID NO:960), rs535908 (C/T) (SEQ ID NO:1470), rs510628 (A/T) (SEQ ID NO:1461) defining the haplotype C C A (or nucleotides from the complementary strand);
t) rs2706218 (C/T) (SEQ ID NO:1209), rs897167 (C/T) (SEQ ID NO:1862), rs10506333 (C/G) (SEQ ID NO:382), rs998401 (C/T) (SEQ ID NO:2038), and rs3741673 (A/G) (SEQ ID NO:1304) defining the haplotype T C G C G (or nucleotides from the complementary strand);
u) rs762721 (A/C) (SEQ ID NO:1729), rs10491992 (C/G) (SEQ ID NO:170), rs10491993 (A/G) (SEQ ID NO:171), and rs2213177 (A/G) (SEQ ID NO:1084) defining the haplotype C G A A (or nucleotides from the complementary strand);
v) rs760002 (A/C) (SEQ ID NO:1724), rs10483259 (A/C) (SEQ ID NO:48), rs956163 (C/T) (SEQ ID NO:1999), and rs10483261 (A/G) (SEQ ID NO:50) defining the haplotype C C T G (or nucleotides from the complementary strand);
w) rs2713961 (A/G) (SEQ ID NO:1211), rs10512591 (A/G) (SEQ ID NO:455), rs2344989 (C/T) (SEQ ID NO:1138), and rs2452932 (C/G) (SEQ ID NO:1166) defining the haplotype A A T C (or nucleotides from the complementary strand);
x) rs10502579 (C/T) (SEQ ID NO:317), rs627346 (A/G) (SEQ ID NO:1516), rs10502582 (A/T) (SEQ ID NO:318), and rs10502584 (A/G) (SEQ ID NO:320) defining the haplotype T A A G (or nucleotides from the complementary strand);
y) rs5949853 (A/T) (SEQ ID NO:1489), rs4348668 (A/G) (SEQ ID NO:1377), rs707289 (A/G) (SEQ ID NO:1613), and rs20369 (C/T) (SEQ ID NO:1010) defining the haplotype A G A C (or nucleotides from the complementary strand);
z) rs764198 (A/G) (SEQ ID NO:1735), rs10521816 (A/G) (SEQ ID NO: (SEQ ID NO:609), rs10521817 (C/T) (SEQ ID NO:610), and rs3135496 (C/T) (SEQ ID NO:1279) defining the haplotype G A C T (or nucleotides from the complementary strand);
aa) rs10521773 (C/T) (SEQ ID NO:604), rs2022565 (A/G) (SEQ ID NO:1001), rs1569890 (A/G) (SEQ ID NO:863), rs5977614 (C/T) (SEQ ID NO:1494), and rs10521774 (C/G) (SEQ ID NO:605) defining the haplotype C G G T G (or nucleotides from the complementary strand);

74-101. (canceled)

102. A method for assessing susceptibility or predisposition to AMI in an individual, the method comprising determining alteration of biological activity, expression or transcription levels of one or several of the genes of table 9 in the individual, wherein a difference in expression is indicative of susceptibility to AMI.

103-178. (canceled)

179. The method according to claim 102, the method comprising determining alteration of translation of mRNAs encoded by one or several of the genes of table 9 in the individual, wherein a difference in translation is indicative of susceptibility to AMI.

180. A method for assessing susceptibility or predisposition to AMI in an individual, the method comprising determining alteration of structure of one or several ot the polypeptides encoded by one or several of the genes of table 9 in the individual, wherein a difference in polypeptide structure of one or several ot the polypeptides is indicative of susceptibility to AMI.

181. A method for assessing susceptibility or predisposition to AMI in an individual, the method comprising determining alteration of amount of one or several ot the metabolites of a polypeptide or polypeptides encoded by one or several of the genes of table 9 in the individual, wherein a difference in amount of one or several ot the metabolites is indicative of susceptibility to AMI.

182. The method according to claim 1, wherein the personal and clinical information, i.e. non-genetic information, concerns age, gender, behaviour patterns and habits, biochemical measurements, clinical measurements, obesity, the family history of CHD, cerebrovascular disease, other cardiovascular disease, hypercholesterolemia, obesity and diabetes, waist-to-hip circumference ratio (cm/cm), socioeconomic status, psychological traits and states, and the medical history of the subject.

183. The method according to claim 182, wherein the behaviour patterns and habits include tobacco smoking, physical activity, dietary intakes of nutrients, alcohol intake and consumption patterns and coffee consumption and quality.

184. The method according to claim 182, wherein the biochemical measurements include determining blood, serum or plasma VLDL, LDL, HDL or total cholesterol or triglycerides, apolipoprotein (a), fibrinogen, ferritin, transferrin receptor, C-reactive protein, glucose, serum or plasma insulin concentration.

185. The method according to claim 182, wherein the non-genetic measurements are those presented in table 11.

186. The method according to claim 182, wherein the non-genetic information contains the mean blood pressure of the subject and the family history of arrhythmia.

187. The method according to claim 182 further comprising a step of calculating the risk of AMI using a logistic regression equation as follows:
Risk of AMI=[1+e^{(a+Σ(bi*Xi)}]⁻¹, where e is Napier's constant, Xᵢ are variables associated with the risk of AMI, bi are coefficients of these variables in the logistic unction, and a is the constant term in the logistic function.

188. The method according to claim 187, wherein a and bi are determined in the population in which the method is to be used.

189. The method according to claim 187, wherein Xi are selected among the variables that have been measured in the population in which the method is to be used.

190. The method according to claim 187, wherein Xi are selected among the SNP markers of tables 3, 4, 5, 7, 8, 10 or 11, among haplotype regions and haplotypes of tables 4, 5, 7, 8, 10 or 11 and among non-genetic variables of the invention.

191. The method according to claim 187, wherein bi are between the values of −20 and 20 and/or wherein Xᵢ can have values between −99999 and 99999 or are coded as 0 (zero) or 1 (one).

192. The method according to claim 187, wherein i are between the values 0 (none) and 100,000.

193. The method according to claim 1, wherein subject's short term, median term, and/or long term risk of AMI is predicted.

194. A method for identifying compounds useful in prevention or treatment of CHD such as AMI comprising determining the effect of a compound on a biological activity of one or several polypeptides encoded by AMI risk genes of table 9 in living cells; wherein a compound altering biological activity of the polypeptide(s) is considered useful in prevention or treatment of CHD such as AMI.

195. The method according to claim 194 comprising determining the effect of a compound on biological networks and/or metabolic pathways related to one or several polypeptides encoded by AMI risk genes of table 9 in living cells; wherein a compound altering activity of one or several said biological networks and/or metabolic pathways are considered useful in prevention or treatment of CHD such as AMI.

196. A method for prevention or treatment of CHD such as AMI comprising administering to a mammalian subject in need of such treatment an effective amount of a compound in a pharmaceutically acceptable carrier enhancing or reducing biological activity of one or several polypeptides encoded by AMI risk genes of table 9; and/or enhancing or reducing activity of one or several biological networks and/or metabolic pathways related to said polypeptides.

197. The method according to claim 196 comprising administering to a mammalian subject in need of such treatment an effective amount of a compound in a pharmaceutically acceptable carrier enhancing or reducing expression of one or several AMI risk genes of table 9; and/or enhancing or reducing the expression of one or several genes in biological networks and/or metabolic pathways related to polypeptides encoded by said AMI risk genes.

198. The method according to claim 196 comprising administering to a mammalian subject in need of such treatment an effective amount of a compound in a pharmaceutically acceptable carrier enhancing or reducing activity of one or several pathophysiological pathways involved in cardiovascular diseases and related to polypeptides encoded by AMI risk genes of table 9.

199. The method according to claim 196, said method comprising the steps of:
a) providing a biological sample taken from a subject;
b) determining the nucleotides present in one or several of the polymorphic sites associated with altered expression and/or biological activity and present in AMI risk genes of table 9 in said individual's nucleic acid; and
c) combining polymorphic site genotype data to select effective therapy for treating CHD such as AMI in said subject.

200. The method according to claim 196, said method comprising the steps of:
a) providing a biological sample taken from a subject;
b) determining expression of one or several AMI risk genes of table 9 and/or determining biological activity of one or several polypeptides encoded by the AMI risk genes of table 9 in said individual's sample; and
c) combining the expression and/or biological activity data to select effective therapy for treating CHD such as AMI in said subject.

201. The method according to claim 196, wherein said treatment is gene therapy or gene transfer.

202. The method according to claim 201, wherein said treatment comprises the transfer of one or several AMI risk genes of table 9 or variants, fragments or derivatives thereof.

203. The method according to claim 201, wherein said AMI risk genes of table 9 or variants, fragments or derivatives thereof are associated with reduced risk of CHD such as AMI.

204. The method according to claim 201, wherein said treatment comprises treating regulatory regions and/or gene containing region of one or more AMI risk genes of table 9 or variants, fragments or derivatives thereof in somatic cells of said subject.

205. The method according to claim 201, wherein said treatment comprises treating regulatory regions and/or gene containing region of one or more AMI risk genes of table 9 or variants, fragments or derivatives thereof in stem cells.

206. The method according to claim 205, wherein said treatment comprises treating regulatory regions and/or gene containing region of one or more AMI risk genes of table 9 or variants, fragments or derivatives thereof in stem cells in tissues affected by cardiovascular diseases.

207. The method according to claim 196, wherein said compound is a recombinant polypeptide encoded by an AMI risk gene of table 9 or variant, fragment or derivative thereof.

208. The method according to claim 196, wherein said treatment is based on siRNA hybridising to mRNA and/or to hnRNA of an AMI risk gene of table 9.

209. The method according to claim 196, wherein said treatment is based on siRNA hybridising to mRNA and/or to hnRNA of one or several genes in biological networks and/or metabolic pathways related to polypeptides encoded by said AMI risk genes of table 9.

210. The method according to claim 198, wherein said method of treating is a dietary treatment or a vaccination.

211. A method for treating a human subject suffering from CHD such as AMI, or having an increased risk of CHD such as AMI said method comprising a therapy restoring, at least partially, the observed alterations in biological activity of one or several polypeptides encoded by AMI risk genes of table 9 in said subject, when compared with CHD free healthy subjects.

212. The method according to claim 211 comprising a therapy restoring, at least partially, the observed alterations in expression of one or several AMI risk genes of table 9 in said subject, when compared with AMI free healthy subjects.

213. A method for monitoring the effectiveness of treatment of CHD such as AMI in a human subject the method comprising measuring mRNA levels of AMI risk genes of table 9, and/or levels of polypeptides encoded by said AMI risk genes, and/or biological activity of polypeptides encoded by said AMI risk genes in a biological sample taken from said subject; alteration of mRNA levels or polypeptide levels or biological activity of a polypeptide following treatment being indicative of the efficacy of the treatment.

214. A method for predicting the effectiveness of a given therapeutic for CHD such as AMI prevention or treatment in a given individual comprising screening for the presence or absence of the AMI associated SNP markers, haplotypes or haplotype regions in one or several of the AMI risk genes of claim 15.

215. A method for predicting the effectiveness of a given therapeutic for CHD such as AMI prevention or treatment in a given individual, the method comprising the steps of
a) providing a biological sample taken from a subject
b) determining the nucleotides present in one or several of the polymorphic sites as set forth in tables 3, 4, 5, 6, 7, 8, 10 and 11 in said individual's nucleic acid
c) combining the SNP marker data to predict the effectiveness of a given therapeutic in an individual for CHD such as AMI prevention or treatment.

216. A method for diagnosing of a subtype of AMI in an individual having AMI, the method comprising the steps of:
a) providing a biological sample taken from a subject;
b) determining the nucleotides present in one or several of the SNP markers as set forth in tables 3, 4, 5, 6, 7, 8, 10 and 11 in said individual's nucleic acid; and
c) combining the SNP marker data to assess the subtype of AMI of an individual.

217. A method for diagnosing of a subtype of AMI in an individual having AMI, the method comprising the steps of:
a) providing a biological sample taken from said individual;
b) collecting personal and clinical information of said individual;
c) determining the nucleotides present in one or several of the SNP markers as set forth in tables 3, 4, 5, 6, 7, 8, 10 and 11 in said individual's nucleic acid; and
d) combining the SNP marker data with personal and clinical information to assess the subtype of AMI of an individual.

218. The method according to claim 217, wherein said one or several SNP markers reside within an AMI risk gene or genes as set forth in table 9.

219. The method according to claim 217, wherein the AMI risk genes reside in the genome region which is defined by the haplotype pattern mining analysis, the genes and regions set forth in tables 4, 5, 6, 7, 8, 10 and 11.

220. The method according to claim 217, wherein the polymorphic sites are associated with the haplotype regions, haplotypes or SNP markers defining the haplotypes set forth in tables 4, 5, 6, 7, 8, 10 and 11.

221. The method according to claim 217, wherein the polymorphic sites are in complete linkage disequilibrium with the haplotype regions, haplotypes or SNP markers defining the haplotypes set forth in tables 4, 5, 6, 7, 8, 10 and 11.

222. The method according to claim 217, wherein the polymorphic sites are in complete linkage disequilibrium in the population in which the said method is used.

223. The method according to claim 1 further comprising a step of combining non-genetic information with the results obtained according to any one of claims 41 to 61 or 63 to 68.

224. The method according to claim 223, wherein the non-genetic information concerns age, gender, behaviour patterns and habits, biochemical measurements, clinical measurements, obesity, the family history of CHD, cerebrovascular disease, other cardiovascular disease, hypercholesterolemia, obesity and diabetes, waist-to-hip circumference ratio (cm/cm), socioeconomic status, psychological traits and states, and the medical history of the subject.

225. The method according to claim 223, wherein the behaviour patterns and habits include tobacco smoking, physical activity, dietary intakes of nutrients, alcohol intake and consumption patterns and coffee consumption and quality.

226. The method according to claim 223, wherein the biochemical measurements include determining blood, serum or plasma VLDL, LDL, HDL or total cholesterol or triglycerides, apolipoprotein (a), fibrinogen, ferritin, transferrin receptor, C-reactive protein, glucose, serum or plasma insulin concentration.

227. The method according to claim 223, wherein the non-genetic measurements are those presented in table 11.

228. The method according to claim 223, wherein the non-genetic information contains the mean blood pressure of the subject and the family history of arrhythmia.

229. A method for measuring AMI risk gene product protein expression, production or concentration in a biological sample taken from a subject, wherein said AMI risk gene is as defined in table 9, the method comprising the steps of:
a) providing a biological sample taken from a subject to be tested,
b) detecting the expression, production or concentration of said protein in said sample,
wherein altered expression, production or concentration indicates an altered risk of cardiovascular disease in said subject

230. A test kit based on a method according to claim 1 for assessment of an altered risk of or susceptibility for AMI in a subject.

231. A test kit for determining the nucleotides present in one or several of the SNP markers as set forth in tables 3, 4, 5, 6, 7, 8, 10 and 11 in said individual's nucleic acid for assessment of an altered risk of AMI in a subject.

232. A test kit for determining the nucleotides present in one or several of the SNP markers as set forth in tables 3, 4, 5, 6, 7, 8, 10 and 11 in said individual's nucleic acid for assessment of an altered risk of AMI in a subject, containing:
a) reagents and materials for assessing nucleotides present in one or several SNP markers as set forth in tables 3, 4, 5, 6, 7, 8, 10 and 11; and
b) software to interpret the results of the determination.

233. The test kit of claims 230 further comprising PCR primer set for amplifying nucleic acid fragments containing one or several SNP markers as set forth in tables 3, 4, 5, 6, 7, 8, 10 and 11 from the nucleic acids of the subject.

234. The test kit of any one of claims 230 comprising a capturing nucleic acid probe set specifically binding to one or several SNP markers present in AMI associated markers and haplotype regions as set forth in tables 3, 4, 5, 6, 7, 8, 10 and 11.

235. The test kit of any one of claims 230 comprising a microarray or multiwell plate to assess the genotypes.

236. The test kit of any one of claims 230 comprising a questionnaire for obtaining patient information concerning age, gender, height, weight, waist and hip circumference, skinfold and adipose tissue thicknesses, the proportion of adipose tissue in the body, the family history of diabetes and obesity, the medical history concerning AMI.

237. A test kit for detecting the presence of SNP markers in one or several of AMI risk genes as set forth in table 9 in a biological sample, wherein said SNP markers are more frequently present in a biological sample of a subject susceptible to AMI compared to a sample from a subject not susceptible to AMI, the kit comprising:
a) reagents and materials for assessing nucleotides present in SNP markers in one or several of AMI risk genes as set forth in table 9; and
b) software to interpret the results of the determination.

238. The test kit of claim 237 further comprising PCR primer set for amplifying nucleic acid fragments containing said SNP markers from AMI risk genes as set forth in table 9 from the nucleid acids of the subject.

239. The test kit of claim 237 comprising a capturing nucleic acid probe set specifically binding to one or several SNP markers present in AMI risk genes as set forth in table 9.

240. The test kit of claim 237 comprising a microarray or multiwell plate to assess the genotypes.

241. The test kit of claim 237 comprising a questionnaire for obtaining patient information concerning age, gender, height, weight, waist and hip circumference, skinfold and adipose tissue thicknesses, the proportion of adipose tissue in the body, the family history of diabetes and obesity, the medical history concerning AMI.

242. A test kit based on a method according to claim 211.

243. The test kit of claim 242 further comprising PCR primer set for amplifying nucleic acid fragments containing said SNP markers from AMI risk genes as set forth in tables 3, 4, 5, 6, 7, 8, 10 and 11 from the nucleid acids of the subject.

244. The test kit of claim 242 comprising a capturing nucleic acid probe set specifically binding to one or several SNP markers present in AMI risk genes as set forth in tables 3, 4, 5, 6, 7, 8, 10 and 11.

245. The test kit of claim 242 comprising a microarray or multiwell plate to assess the genotypes.

246. The test kit of claim 242 comprising a questionnaire for obtaining patient information concerning age, gender, height, weight, waist and hip circumference, skinfold and adipose tissue thicknesses, the proportion of adipose tissue in the body, the family history of diabetes and obesity, the medical history concerning AMI.

247. The test kit of claim 230 further comprising a marker set to assess the ancestry of an individual.

248. The test kit of claim 247 comprising a SNP marker set to assess the ancestry of an individual.

249. The test kit of claim 247 comprising a microsatellite marker set to assess the ancestry of an individual.

250. A method of claim 1 further comprising a marker set to assess the ancestry of an individual.

251. A method of claim 1 comprising a SNP marker set to assess the ancestry of an individual.

252. A method of claim 1 comprising a microsatellite marker set to assess the ancestry of an individual.
